(19) United States
(12) Patent Application Publication
(10) Pub. No.: US 2009/0247504 A1
(43) Pub. Date: Oct. 1, 2009

(54) Indazole Derivatives for Treatment of Alzheimer's Disease

(76) Inventors: Ian Churcher, Herts (GB); Hedaythul Choudhury, Hert (GB); Neil Wilkie, Dundee (GB); Peter Hunt, Horsham (GB); Richard Jelley, Newbury (GB); Alan Nadin, Hertfordshireh (GB); Carmel Nanthakumar, Harrow (GB); Peter Simpson, Macclesfield (GB)

Correspondence Address: MERCK AND CO., INC, P O BOX 2000, RAHWAY, NJ, 07065-0907, US

(21) Appl. No.: 12/223,415

(22) PCT Filed: Feb. 2, 2007
(86) PCT No.: PCT/GB2007/050048

§ 371 Date: Dec. 9, 2008

(30) Foreign Application Priority Data
Feb. 3, 2006 (GB) 0602178.6

(51) Int. Cl. A61K 31/416; A61K 31/5377; A61K 31/397; C07D 231/56; C07D 413/14; A61P 25/28

(52) U.S. Cl. 514/210.21; 514/406; 514/233.8; 548/362.5; 544/140

(57) ABSTRACT

Compounds of formula (IA) and (IB) are inhibitors of MARK, and hence are suitable for treatment of Alzheimer's disease.

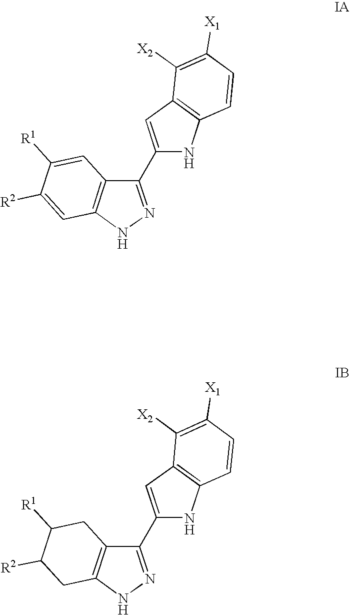

This invention relates to methods and materials for the treatment or prevention of neurodegenerative diseases such as Alzheimer's disease. In particular, there is disclosed a particular class of indazole derivatives which selectively inhibit microtubule affinity regulating kinase (MARK).

Alzheimer's disease (AD) is the most common cause of dementia in the elderly and is characterised by a decline in cognitive function, that progresses slowly and results in symptoms such as memory loss and disorientation. Death occurs, on average, 9 years after diagnosis. The incidence of AD increases with age, so that while about 5% of people over the age of 70 are sufferers, this FIGURE increases to 20% of those over 80 years old.

Existing treatments exclusively target the primary symptoms of AD. Diseased neurons may release insufficient or excessive amounts of particular neurotransmitters, and so current drugs are aimed at increasing neurotransmitter levels or at reducing the stimulation of nerve cells by neurotransmitters. Although these drugs provide some improvement in the symptoms of AD, they fail to address the underlying cause of the disease.

The classic clinical and neuropathological features of AD consist of senile or neuritic plaques and tangled bundles of fibers (neurofibrillary tangles) [Verdile, G., et al, Pharm. Res. 50:397-409 (2004)]. In addition, there is a severe loss of neurons in the hippocampus and the cerebral cortex. Neuritic plaques are extracellular lesions, consisting mainly of deposits of β-amyloid peptide (Aβ), surrounded by dystrophic (swollen, damaged and degenerating) neurites and glial cells activated by inflammatory processes. In contrast, neurofibrillary tangles (NFTs) are intracellular clusters composed of a hyperphosphorylated form of the protein tau, which are found extensively in the brain (e.g. mainly in cortex and hippocampus in AD). Tau is a soluble cytoplasmic protein which has a role in microtubule stabilisation. Excessive phosphorylation of this protein renders it insoluble and leads to its aggregation into paired helical filaments, which in turn form NFTs.

The amyloid cascade hypothesis proposes that abnormal accumulation of Aβ peptides, particularly Aβ42, initiates a cascade of events leading to the classical symptoms of AD and ultimately, to the death of the patient. There is strong evidence [e.g. Rapoport, M., et al (2002) Proc. Natl. Acad. Sci. USA 99:6364-6369] that dysregulation of tau function is a key step in the cascade of Alzheimer's disease pathology leading ultimately to neuronal death. Furthermore, tau mutations and NFTs are found in other dementias in which Aβ pathology is absent, such as frontotemporal dementia, Pick's disease and parkinsonism linked to chromosome 17 (FTDP-17) [Mizutani, T. (1999) Rinsho Shikeigaku 39: 1262-1263]. Also, in AD the frequency of NFTs correlates to the degree of dementia better than that of senile plaques [Arriagada, P. V., et al (1992) Neurology 42:631-639], while significant numbers of amyloid plaques are often found in the brains of non-demented elderly people, suggesting that amyloid pathology on its own is not sufficient to cause dementia. For these reasons, normalisation of tau function (in particular prevention of hyperphosphorylation) is seen as a desirable therapeutic goal for the treatment of AD and other dementing conditions.

Tau is a 352-441 amino acid protein encoded by the Mapt (Microtubule-associated protein tau) gene which is widely expressed in the central nervous system (CNS) with localisation primarily in axons [Binder et al J. Cell Biol. 1985, 101(4), 1371-1378]. The major function of tau is regulation of the stability of microtubules (MTs), intracellular structural components comprised of tubulin dimers which are integral in regulating many essential cellular processes such as axonal transport and elongation as well as generation of cell polarity and shape. Tau binding to tubulin is a key factor in determining the rates of polymerisation/depolymerisation (termed dynamic instability) of MTs, and tau is therefore key to the regulation of many essential cellular processes [see, for example, Butner, K. A., Kirschner, M. W. (1991) J. Cell. Biol. 115: 717-730].

Tau is a basic protein with numerous serine and threonine residues, many of which are susceptible to phosphorylation. While normal tau has two to three phosphorylated amino acid residues, hyperphosphorylated tau found in AD and other tauopathies typically has eight or nine phosphorylated residues. A variety of kinases promote phosphorylation of these sites, including proline-directed kinases such as glycogen synthase kinase 3β (GSK3β) and cyclin dependent kinase 5 (cdk5), and non-proline-directed kinases such as protein kinase A (PKA) and calmodulin (CaM) kinase II, which phosphorylate tau at Lys-(Ile/Cys)-Gly-Ser sequences, also known as KXGS motifs. One KXGS motif is found in each of the MT binding repeats. Phosphorylation at these sites is important for the regulation of tau-MT binding and while the degree of phosphorylation is normally low, it has been shown to be increased in brain tissue from AD patients. Phosphorylation of one particular residue within the KXGS motifs, Ser-262 has been shown to be elevated in tau protein extracted from the NFTs in AD [Hasegawa, M. et al (1992) J. Biol. Chem 267:17047-17054] and phosphorylation at this site also appears to dramatically reduce MT binding [Biernat, J. et al. (1993) Neuron 11: 153-163].

Nishimura et al. [Cell 116: 671-682 (2004)] demonstrated that overexpression of the kinase PAR-1 in Drosophila led to enhanced tau-mediated toxicity and an increase in the phosphorylation of tau on Ser-262, Ser-356, and other amino acid residues, including sites phosphorylated by GSK3β and Cdk5. Their findings suggest that PAR-1 kinase acts as a master kinase during the process of tau hyperphosphorylation, with the phosphorylation of the Ser-262 and Ser-356 sites being a prerequisite for the subsequent phosphorylation at downstream sites by other kinases.

The mammalian ortholog of PAR-1 is microtubule affinity-regulating kinase (MARK). There are four MARK isoforms and these form part of the AMP-dependent protein kinase (AMPK) family. Like PAR-1, MARK is thought to phosphorylate tau, perhaps in response to an external insult, such as the disruption of Ca²⁺ homeostasis caused by Aβ, priming it for further phosphorylation events. It is not clear whether the phosphorylation of tau by MARK leads directly to its detachment from MTs or the subsequent phosphorylation events cause detachment. The resulting unbound, hyperphosphorylated tau is delocalised to the somatodendritic compartment and is then cleaved by caspases to form fragments prone to aggregation [Drewes, G. (2004). Trends Biochem. Sci 29:548-555; Gamblin, T. C., et al, (2003) Proc. Natl. Acad. Sci. U.S.A. 100: 10032-10037]. These aggregates can grow into filaments, which are potentially toxic, eventually forming the NFTs found in AD.

For these reasons, it is proposed that MARK inhibitors will enable the prevention or amelioration of neurodegeneration in AD and other tauopathies.

WO 01/02369, WO 03/024969 and US 2004/0242559 disclose various 3-(indol-2-yl)indazole derivatives as inhibitors of various kinases (mainly tyrosine kinase inhibitors), implicated in cell proliferative processes, but there is no disclosure of utility as MARK inhibitors or in the treatment or prevention of tauopathies.

WO 00/69846 discloses a class of tetrahydroindazole derivatives as inhibitors of cyclin-dependent kinases, also useful in control of cell proliferation, but does not disclose or suggest compounds relevant to the present invention, or inhibition of MARK.

According to the invention, there is provided the use, for the manufacture of a medicament for treatment or prevention of a neurodegenerative disease associated with hyperphosphorylation of tau, of a compound according to formula IA or formula IB:

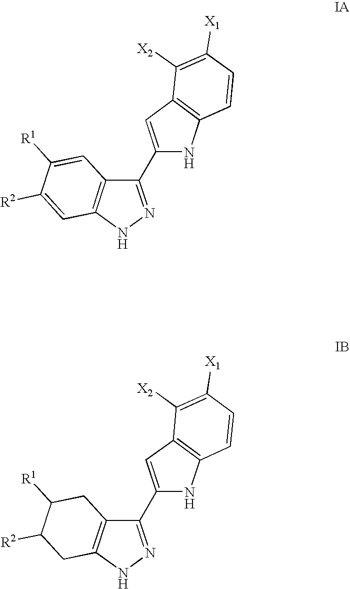

or a pharmaceutically acceptable salt or hydrate thereof: wherein

one of R¹ and R² represents H, halogen or C₁₋₄alkyl and the other is selected from H, halogen, CN, NO₂, CF₃, OR⁵, N(R⁵)₂, aryl which optionally bears up to 3 substituents selected from halogen, CN, NO₂, CF₃, OR⁵, C₁₋₄alkyl, hydroxyC₁₋₄alkyl, CO₂R⁵ and CON(R⁵)₂, and non-aromatic hydrocarbon of up to 6 carbon atoms which is optionally substituted with halogen, CN, CF₃ or OR⁵;

or in formula IB R¹ or R² may represent oxo;

one of X1 and X2 represents H and the other represents L-OR³ or L-NR³R⁴; where L represents a bond or an alkylene group of up to 4 carbon atoms which optionally bears an oxo substituent;

R³ represents H or nonaromatic hydrocarbon of up to 10 carbon atoms, optionally substituted with halogen, CN, CF₃, OR⁵, N(R⁵)₂ or NR⁵COC₁₋₄alkyl;

or R³ represents aryl, arylC₁₋₄alkyl, C-heterocyclyl or C-heterocyclylC₁₋₄alkyl, any of which optionally bears up to 3 substituents selected from halogen, CN, CF₃, OR⁵, C₁₋₄alkyl, aryl or arylC₁₋₄alkyl;

R⁴ represents H or C₁₋₄alkyl; or R³ and R⁴ together complete a mono- or bicyclic heterocyclic ring system of up to 10 members which optionally bears up to 3 substituents selected from halogen, CN, CF₃, C₁₋₄alkyl, oxo, OR⁵, N(R⁵)₂, (5)₂NC₁₋₄alkyl, COC₁₋₄alkyl, aryl, arylC₁₋₄alkyl, C-heterocyclyl and C-heterocyclylC₁₋₄alkyl, said aryl, arylC₁₋₄alkyl, C-heterocyclyl and C-heterocyclylC₁₋₄alkyl themselves optionally bearing up to 3 substituents selected from halogen, CN, CF₃, C₁₋₄alkyl and OR⁵;

R⁵ represents H or C₁₋₄alkyl, or two R⁵ groups attached to the same nitrogen atom may complete a heterocyclic ring of 5 or 6 members optionally bearing a substituent selected from halogen, oxo, CF₃ and C₁₋₄alkyl;

where “aryl” refers to phenyl, naphthyl or optionally benzofused 5- or 6-membered heteroaryl, and “C-heterocyclyl” refers to a 5- or 6-membered nonaromatic ring in which the attachment point is a carbon atom and in which from 1 to 3 of the ring atoms are independently selected from N, O and S.

The invention further provides a method for treatment or prevention of a neurodegenerative disease associated with hyperphosphorylation of tau in a human patient, said method comprising administering to that patient an effective amount of a compound of formula IA or IB as defined above, or a pharmaceutically acceptable salt or hydrate thereof.

Neurodegenerative diseases associated with hyperphosphorylation of tau include AD, frontotemporal dementia, Pick's disease and parkinsonism linked to chromosome 17 (FTDP-17).

Compounds of formula IB as defined above and the pharmaceutically acceptable salts and hydrates thereof are believed to be novel, and constitute a further aspect of the invention.

In a further aspect, the invention provides a pharmaceutical composition comprising a compound of formula IB or a pharmaceutically acceptable salt or hydrate thereof and a pharmaceutically acceptable carrier.

As used herein, the expression “hydrocarbon group” refers to groups consisting solely of carbon and hydrogen atoms. Such groups may comprise linear, branched or cyclic structures, singly or in any combination consistent with the indicated maximum number of carbon atoms, and may be saturated or unsaturated, including aromatic when the indicated maximum number of carbon atoms so permits unless otherwise indicated.

As used herein, the expression “C₁₋ₓalkyl” where x is an integer greater than 1 refers to straight-chained and branched alkyl groups wherein the number of constituent carbon atoms is in the range 1 to x. Particular alkyl groups are methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₁₋₆alkoxy” are to be construed in an analogous manner. Most suitably, the number of carbon atoms in such groups is not more than 6.

The term “halogen” as used herein includes fluorine, chlorine, bromine and iodine.

The expression “C₃₋₆cycloalkyl” as used herein refers to nonaromatic monocyclic hydrocarbon ring systems comprising from 3 to 6 ring atoms. Examples include cyclopropyl, cyclobutyl, cyclopentyl and cyclohexyl.

For use in medicine, the compounds of formula I may be in the form of pharmaceutically acceptable salts. Other salts may, however, be useful in the preparation of the compounds of formula I or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, benzenesulphonic acid, fumaric acid, maleic acid, succinic acid, acetic acid, benzoic acid, oxalic acid, citric acid, tartaric acid, carbonic acid or phosphoric acid. Alternatively, where the compound of the invention carries an acidic moiety, a pharmaceutically acceptable salt may be formed by neutralisation of said acidic moiety with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

When the compounds useful in the invention have one or more asymmetric centres, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

When a compound useful in the invention is capable of existing in tautomeric keto and enol forms, both of said forms are considered to be within the scope of the invention.

A nitrogen atom forming part of a heteroaryl ring may be in the form of the N-oxide. A sulphur atom forming part of a nonaromatic heterocycle may be in the form of the S-oxide or S,S-dioxide.

A heteroaryl group may be attached to the remainder of the molecule via a ring carbon or a ring nitrogen, provided that this is consistent with preservation of aromaticity.

In formulae IA and IB, at least one of R¹ and R² represents H, halogen or C₁₋₄alkyl, and in a particular embodiment at least one of R¹ and R² represents H. In a further embodiment R¹ and R² are both H.

When R¹ is H, R² is typically selected from H, halogen (especially Cl or Br), CN, NO₂, N(R⁵)₂ (such as NH₂), C₁₋₄alkyl (such as methyl, ethyl or propyl), hydroxyC₁₋₄alkyl (such as 2-hydroxyethyl or 3-hydroxypropyl), C₂₋₄alkenyl (such as vinyl or allyl), C₂₋₄alkynyl (such as ethynyl or propynyl) and hydroxyC₂₋₄alkynyl (such as 3-hydroxypropynyl).

When R² is H, R¹ is typically selected from H, halogen (especially Cl or Br), CN, NO₂, N(R⁵)₂ (such as NH₂), OH and optionally-substituted aryl, where “aryl” is as defined previously. Examples of aryl groups represented by R¹ include phenyl, pyridyl, furanyl, pyrazolyl, isoquinolinyl, isoxazolyl, pyrimidinyl, tetrazolyl, pyridazinyl, triazolyl, pyrazinyl, thiophenyl, thiazolyl, isothiazolyl, Examples of optional substituents on said aryl groups include C₁₋₄alkyl (such as methyl), hydroxyC₁₋₄alkyl (such as hydroxymethyl), C₁₋₄alkoxy (such as methoxy), OH, CN, halogen (such as F or Cl), CO₂H, C₁₋₄alkoxycarbonyl (such as methoxycarbonyl or ethoxycarbonyl), and CON(R⁵)₂ (such as CONH₂ or CONMe₂), In a particular embodiment, said aryl group bears not more than 2 optional substituents. Particular examples of aryl groups represented by R¹ include phenyl, 2-pyridyl, 3-pyridyl, 4-pyridyl, 6-methoxy-3-pyridyl, 2-methoxy-3-pyridyl, 3-cyanophenyl, furan-3-yl, 3-hydroxyphenyl, pyrazol-3-yl, pyrazol-4-yl, 1-methylpyrazol-4-yl, isoquinolin-3-yl, isoquinolin-4-yl, 4-fluoro-2-methoxyphenyl, 3-carbamoylphenyl, 3-(N,N-dimethyl)carbamoylphenyl, 3-carboxyphenyl, 3,5-dimethylisoxazol-4-yl, 2-methoxypyrimidin-5-yl, pyrimidin-5-yl, 5-carboxy-3-pyridyl, 5-ethoxycarbonyl-3-pyridyl, 3-(hydroxymethyl)phenyl, tetrazol-5-yl, pyridazin-4-yl, 1,2,4-triazol-2-yl, pyrazin-2-yl, 3-fluorophenyl, thiophen-3-yl, isothiazol-4-yl, thiazol-5-yl, 6-fluoro-3-pyridyl, and 6-hydroxy-3-pyridyl (or the keto tautomer thereof).

In a particular embodiment, R² is H and R¹ is 5- or 6-membered heteroaryl, in particular 3-pyridyl, 1-methylpyrazol-4-yl or isothiazol-4-yl.

In formulae IA and IB, one of X1 and X2 represents H and the other represents L-OR³ or L-NR³R⁴ where L, R³ and R⁴ are as defined previously. In a particular embodiment, X2 represents H.

Typical identities for L include a bond, CO and CH₂. In a particular embodiment, L represents CH₂.

Typical identities for X1 or X2 include CH₂NR³R⁴, CONR³R⁴, CO₂R³ (such as CO₂H), CH₂OR³ (such as CH₂OH) and OR³. In a particular embodiment, X1 or X2 represents CH₂NR³R⁴.

When R³ represents a hydrocarbon group, this is typically selected from C₁₋₆alkyl groups (such as methyl, ethyl, propyl or butyl), C₂₋₆alkenyl groups (such as allyl), C₃₋₆cycloalkyl groups (such as cyclopentyl or cyclohexyl) and C₃₋₆cycloalkylC₁₋₄alkyl groups (such as cyclohexylmethyl). Any of said hydrocarbon groups may be substituted with halogen, CN, CF₃, OR⁵, N(R⁵)₂ or NR⁵COC₁₋₄alkyl where R⁵ is as defined previously. Preferred substituents include OR⁵, N(R⁵)₂ and NR⁵COC₁₋₄alkyl, in particular N(R⁵)₂ and NR⁵COC₁₋₄alkyl. Typically, R⁵ represents H or methyl, or two R⁵ groups attached to the same nitrogen atom complete a heterocyclic ring of 5 or 6 members optionally bearing a substituent selected from halogen, oxo, CF₃ and C₁₋₄alkyl. Examples of heterocyclic groups represented by N(R⁵)₂ include piperidin-1-yl, morpholin-4-yl, 4-methylpiperazin-1-yl, pyrrolidin-1-yl and 2-oxopyrrolidin-1-yl.

When R³ represents aryl or arylC₁₋₄alkyl, said aryl is typically optionally-substituted phenyl, pyridyl or 5-membered heteroaryl (such as thiazole or triazole). Preferred substituents include C₁₋₄alkyl (such as methyl) and C₁₋₄alkoxy (such as methoxy). Examples include 4-pyridylmethyl, 4-methoxybenzyl, 1-phenylethyl, 2-methylthiazol-4-ylmethyl and 5-methyl-1,3,4-triazol-2-ylmethyl.

When R³ represents C-heterocyclyl or C-heterocyclylC₁₋₄alkyl, said heterocyclic moiety is typically selected from optionally-substituted pyrrolidine, piperidine, 1,1-dioxotetrahydrothiophene and morpholine. Examples include 1-methylpiperidin-4-yl, piperidin-4-ylmethyl, 1-methylpyrrolidin-3-ylmethyl, 1-methylpiperidin-4-ylmethyl, 1-methylpiperidin-3-ylmethyl, 1-methylpiperidin-2-ylmethyl, 1-methyl-3-benzylpiperidin-4-ylmethyl, 1-(4-pyridylmethyl)piperidin-4-ylmethyl, 4-benzylmorpholin-2-ylmethyl and 1,1-dioxotetrahydrothiophene-3-yl.

R⁴ typically represents H or methyl; or R³ and R⁴ together complete a mono- or bicyclic heterocyclic ring system of up to 10 members which is optionally substituted as defined previously. Examples of suitable monocyclic ring systems include azetidine, pyrrolidine, piperidine, piperazine, morpholine and thiomorpholine. Examples of suitable bicyclic ring systems include 2,5-diazabicyclo[2,2,1]heptane, 2,7-diazaspiro[4,4]nonane, octahydro[1,2,4]triazolo[4,3-a]pyrazine and 5,6,7,8-tetrahydro[1,2,4]triazolo[4,3-a]pyrazine. Preferred substituents include C₁₋₄alkyl (such as methyl), COC₁₋₄alkyl (such as acetyl), dimethylamino, dimethylaminoC₁₋₄alkyl (such as 2-(dimethylamino)ethyl), OH, and phenyl or benzyl which themselves may be substituted (e.g. with methoxy).

In a subset of the compounds of formulae IA and IB, X1 or X2 represents CH₂NR³R⁴, and NR³R⁴ takes the form:

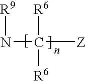

where n is 1, 2, 3 or 4;

Z represents OR⁷ or NR⁷R⁸;

each R⁶ independently represents H, or together with R⁷ represents the atoms necessary to complete a 5- or 6-membered heterocyclic ring, or together with R⁹ represents the atoms necessary to complete a 5- or 6-membered heterocyclic ring, or two R⁶ groups may together represent the atoms necessary to complete a 5- or 6-membered carbocyclic ring;

R⁷ represents H or C₁₋₄alkyl, or together with an R⁶ group represents the atoms necessary to complete a 5- or 6-membered heterocyclic ring, or together with R⁹ represents the atoms necessary to complete a 5- or 6-membered heterocyclic ring;

R⁸ represents H, C₁₋₄alkyl or COC₁₋₄alkyl; or R⁷ and R⁸ may complete a heterocyclic ring of 5 or 6 members optionally bearing a substituent selected from halogen, oxo, CF₃ and C₁₋₄alkyl; and

R⁹ represents H or C₁₋₄alkyl, or together with an R⁶ group represents the atoms necessary to complete a 5- or 6-membered heterocyclic ring, or together with R⁷ represents the atoms necessary to complete a 5- or 6-membered heterocyclic ring;

provided that when R⁷ and R⁹ complete a ring, n is 2 and all four R⁶ groups are H; and that when n is 1, both the R⁶ groups are H.

A subset of the compounds useful in the invention consists of the compounds of formula II:

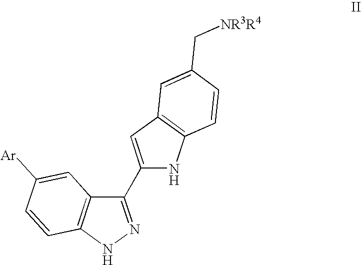

and the pharmaceutically acceptable salts and hydrates thereof;
wherein Ar represents phenyl, naphthyl or optionally benzofused 5- or 6-membered heteroaryl, any of which optionally bears up to 3 substituents selected from halogen, CN, NO₂, CF₃, OR⁵, C₁₋₄alkyl, hydroxyC₁₋₄alkyl, CO₂R⁵ and CON(R⁵)₂,
and R³, R⁴ and R⁵ have the same definitions as before.

Examples of groups represented by Ar include phenyl, pyridyl, furanyl, pyrazolyl, isoquinolinyl, isoxazolyl, pyrimidinyl, tetrazolyl, pyridazinyl, triazolyl, pyrazinyl, thiophenyl, thiazolyl, isothiazolyl, Examples of optional substituents on Ar include C₁₋₄alkyl (such as methyl), hydroxyC₁₋₄alkyl (such as hydroxymethyl), C₁₋₄alkoxy (such as methoxy), OH, CN, halogen (such as F or Cl), CO₂H, C₁₋₄alkoxycarbonyl (such as methoxycarbonyl or ethoxycarbonyl), and CON(R⁵)₂ (such as CONH₂ or CONMe₂), In a particular embodiment, Ar bears not more than 2 optional substituents. Particular examples of groups represented by Ar include phenyl, 2-pyridyl, 3-pyridyl, 4-pyridyl, 6-methoxy-3-pyridyl, 2-methoxy-3-pyridyl, 3-cyanophenyl, furan-3-yl, 3-hydroxyphenyl, pyrazol-3-yl, pyrazol-4-yl, 1-methylpyrazol-4-yl, isoquinolin-3-yl, isoquinolin-4-yl, 4-fluoro-2-methoxyphenyl, 3-carbamoylphenyl, 3-(N,N-dimethyl)carbamoylphenyl, 3-carboxyphenyl, 3,5-dimethylisoxazol-4-yl, 2-methoxypyrimidin-5-yl, pyrimidin-5-yl, 5-carboxy-3-pyridyl, 5-ethoxycarbonyl-3-pyridyl, 3-(hydroxymethyl)phenyl, tetrazol-5-yl, pyridazin-4-yl, 1,2,4-triazol-2-yl, pyrazin-2-yl, 3-fluorophenyl, thiophen-3-yl, isothiazol-4-yl, thiazol-5-yl, 6-fluoro-3-pyridyl, and 6-hydroxy-3-pyridyl (or the keto tautomer thereof).

In a particular embodiment, Ar in formula II is optionally-substituted 5- or 6-membered heteroaryl, in particular 5- or 6-membered heteroaryl comprising up to 2 ring nitrogens, such as pyridyl (e.g. 3-pyridyl), pyrazinyl, pyrimidinyl (e.g. 5-pyrimidinyl), pyridazinyl (e.g. 4-pyridazinyl), pyrazole (e.g. pyrazol-3-yl, pyrazol-4-yl and 1-methylpyrazol-4-yl), thiazole (e.g. thiazol-5-yl) and isothiazole (e.g. isothiazol-4-yl), especially pyridyl and pyrazole.

In a further particular embodiment, NR³R⁴ in formula II takes the form:

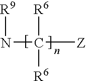

where n, Z, R⁶ and R⁹ are as defined previously. Within this embodiment, Z is very suitably NR⁷R⁸.

Compounds of formula II and the pharmaceutically acceptable salts and hydrates thereof are believed to be novel, and therefore constitute a further aspect of the invention. In yet another aspect, the invention provides a pharmaceutical composition comprising a compound of formula II or a pharmaceutically acceptable salt or hydrate thereof and a pharmaceutically acceptable carrier.

Specific examples of compounds suitable for use in the invention include compounds of formula IB in which X2 is H and the other variables are as shown in table 1:

TABLE 1

| R¹ | R² | X1 |
|---|---|---|
| H | H | CH₂OH |
| H | H | CH₂morpholin-4-yl |
| H | H | CH₂NHCH₂CH₂NH2 |
| ═O | H | CH₂morpholin-4-yl |
| OH | H | CH₂morpholin-4-yl |
| H | ═O | CH₂morpholin-4-yl |
| H | H | CH₂NH-cyclohex-NH₂ |
| H | H | CH₂NHCH₂CH₂NHAc |
| H | H | CH₂NHCH₂CH₂OH |
| H | H | CH₂NHCH₂-4-Py |
| H | H | CH₂NHCH₂CH₂-morpholin-4-yl |
| 3-pyridyl | H | CH₂-morpholin-4-yl |
| 3-pyridyl | H | CH₂NHCH₂CH₂NH₂ |
| 3-pyridyl | H | CH₂NH-cyclohex-NH₂ |
| 3-pyridyl | H | CH₂NHCH₂CH₂NHAc |
| H | H | CH₂NH-(1-Me-piperidin-4-yl) |
| H | H | CH₂NHCH₂CH₂CH₂NMe2 |
| H | H | CH₂NHCH₂-piperidin-1-yl |
| H | H | CH₂NMe₂ |
| H | H | CH₂N(Me)-(1-Me-piperidin-4-yl) |
| H | H | CH₂N(Me)CH₂CH₂CH₂NMe₂ |

“cyclohex” = cyclohexane-1,4,diyl
“Ac” = acetyl
“Py” = pyridyl

Further specific compounds suitable for use in the invention include compounds of formula IA in which X1 is H and the remaining variables are as indicated in Table 2:

TABLE 2

| R¹ | R² | X2 |
|---|---|---|
| H | CN | CH₂NH-[1-(4-PyCH₂)piperidin-4-yl] |
| H | CN | CH₂NHCH₂-(1-Me-piperidin-4-yl) |
| H | CN | CH₂NH-(1-Me-3-benzylpiperidin-4-yl) |
| H | CN | CH₂NH-(4-benzylmorpholin-2-yl) |
| H | CN | 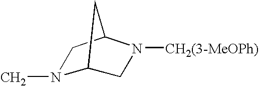 |
| H | CN | 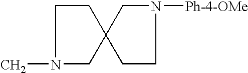 |
| H | CN | 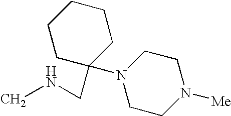 |

Further specific compounds suitable for use in the invention include compounds of formula IA in which X2 is H and the remaining variables are as indicated in Table 3:

TABLE 3

| R¹ | R² | X1 |
|---|---|---|
| H | Cl | CH₂-(4-Ac-piperazin-1-yl) |
| H | Cl | CO₂H |
| H | H | CO-(4-Me-piperazin-1-yl) |
| H | H | CH₂-(4-Ac-piperazin-1-yl) |
| H | H | OCH₂CH₂-morpholin-4-yl |
| H | CN | CH₂NHCH₂CH₂NH₂ |
| H | CN | CH₂NHCH₂-piperidin-4-yl |
| H | CN | CH₂NHCH₂CH₂CH₂NH₂ |
| H | CN | CH₂NH-cyclohex-NH₂ |
| H | CN | CH₂-morpholin-4-yl |
| H | Br | CH₂-morpholin-4-yl |
| H | Me | CH₂-morpholin-4-yl |
| H | Et | CH₂-morpholin-4-yl |
| H | allyl | CH₂-morpholin-4-yl |
| H | nitro | CH₂-morpholin-4-yl |
| H | vinyl | CH₂-morpholin-4-yl |
| H | amino | CH₂-morpholin-4-yl |
| Br | H | CH₂-morpholin-4-yl |
| H | HO(CH₂)₃ | CH₂-morpholin-4-yl |
| H | HO(CH₂)₂ | CH₂-morpholin-4-yl |
| allyl | H | CH₂-morpholin-4-yl |
| H | H | CH₂-morpholin-4-yl |
| H | HOCH₂C≡C | CH₂-morpholin-4-yl |
| nitro | H | CH₂-morpholin-4-yl |
| amino | H | CH₂-morpholin-4-yl |
| CN | H | CH₂-morpholin-4-yl |
| 3-Py | H | CONH-cyclohex-NH₂ |

Further specific compounds suitable for use in the invention include compounds of formula II in which Ar and NR³R⁴ are as indicated in Table 4:

TABLE 4

| Ar | NR³R⁴ |
|---|---|
| 3-Py | morpholin-4-yl |
| 3-Py | NH-cyclohex-NH₂ |
| 6-MeO-3Py | morpholin-4-yl |
| 3-CN—Ph | morpholin-4-yl |
| 2-MeO-3-Py | morpholin-4-yl |
| Ph | morpholin-4-yl |
| 3-furyl | morpholin-4-yl |
| 3-OH—Ph | morpholin-4-yl |
| 4-Py | morpholin-4-yl |
| 1-Me-pyrazol-4-yl | morpholin-4-yl |
| isoquinolin-3-yl | morpholin-4-yl |
| 4-F-2-MeO—Ph | morpholin-4-yl |
| isoquinolin-4-yl | morpholin-4-yl |
| 3-(Me₂NCO)—Ph | morpholin-4-yl |
| 3-(HO₂C)—Ph | morpholin-4-yl |
| 3,5-di-Me-isoxazol-4-yl | morpholin-4-yl |
| 2-MeO-pyrimidin-5-yl | morpholin-4-yl |
| pyrimidin-5-yl | morpholin-4-yl |
| 2-Py | morpholin-4-yl |
| 5-(EtO₂C)-3-Py | morpholin-4-yl |
| 5-(HO₂C)-3-Py | morpholin-4-yl |
| 1-Me-pyrazol-4-yl | NH-cyclohex-NH₂ |
| 3-(HO₂C)—Ph | NH-cyclohex-NH₂ |
| pyrazol-4-yl | morpholin-4-yl |
| 3-Py | NHCH₂CH₂OH |
| 3-Py | NHCH₂CH₂NHAc |
| 3-Py | 4-OH-piperidin-1-yl |
| 3-Py | piperidin-1-yl |
| 3-Py | NHCH₂-4-Py |
| 3-Py | NHCH₂CH₂-morpholin-4-yl |
| 3-Py | 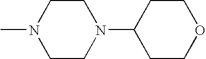 |
| 3-Py | 2-Me-pyrrolidin-1-yl |
| 3-HOCH₂—Ph | morpholin-4-yl |
| 3-(H₂NCO)—Ph | morpholin-4-yl |
| pyrazol-4-yl | NH-cyclohex-NH₂ |
| 3-Py | N(Me)-cyclohex-NH₂ |
| 3-Py | NHCH₂-(4-MeO—Ph) |
| tetrazole-5-yl | morpholin-4-yl |
| pyridazin-4-yl | morpholin-4-yl |
| 3-Py | N(Me)CH₂CH₂NHAc |
| 3-Py | 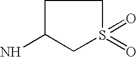 |
| 3-Py | NHCH₂CH₂-2-oxo-pyrrolidin-1-yl |
| 3-Py | 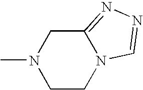 |
| 3-Py | 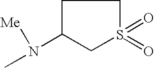 |
| 3-Py | N(Me)CH₂CH₂-2-oxo-pyrrolidin-1-yl |
| pyrazol-4-yl | NHCH₂CH₂NHAc |
| 1,2,4-triazol-1-yl | morpholin-4-yl |
| pyridazin-4-yl | 4-OH-piperidin-1-yl |
| pyridazin-4-yl | NH-cyclohex-NH₂ |
| pyrazin-2-yl | NH-cyclohex-NH₂ |
| 3-F—Ph | NH-cyclohex-NH₂ |
| pyrazol-3-yl | NH-cyclohex-NH₂ |
| thiophen-3-yl | NH-cyclohex-NH₂ |
| isothiazol-4-yl | NH-cyclohex-NH₂ |
| thiazol-5-yl | NH-cyclohex-NH₂ |
| 6-F-3-Py | morpholin-4-yl |
| 6-OH-3-Py | morpholin-4-yl |
| thiophen-3-yl | morpholin-4-yl |
| isothiazol-4-yl | morpholin-4-yl |
| pyrazin-2-yl | morpholin-4-yl |
| 3-Py | NH₂ |
| 3-Py | NHCH₂-(5-Me-thiazol-3-yl) |
| 3-Py | NHCH₂-(5-Me-1,2,4-triazol-3-yl) |
| 3-Py | 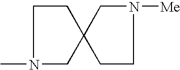 |
| 3-Py | NHCH(Me)Ph |
| 3-Py | 4-Ac-piperazin-1-yl |
| 3-Py | NMe₂ |
| isothiazol-4-yl | piperidin-1-yl |
| 1-Me-pyrazol-4-yl | piperidin-1-yl |
| thiazol-5-yl | piperidin-1-yl |
| isothiazol-4-yl | pyrrolidin-1-yl |
| isothiazol-4-yl | 4-Me-piperazin-1-yl |
| isothiazol-4-yl | azetidin-1-yl |
| isothiazol-4-yl | NHMe |
| isothiazol-4-yl | NH-allyl |
| isothiazol-4-yl | NMe₂ |
| isothiazol-4-yl | NH-cyclohexyl |
| isothiazol-4-yl | thiomorpholin-4-yl |
| isothiazol-4-yl | 2-Me-pyrrolidin-1-yl |
| isothiazol-4-yl | NH-(1-Me-piperidin-4-yl) |
| 3-Py | azetidin-1-yl |
| 3-Py | NHMe |
| 3-Py | NHCH₂CH₂-pyrrolidin-1-yl |
| 3-Py | NHCH₂CH₂CH₂NMe₂ |
| 3-Py | 4-(Me₂N)-piperidin-1-yl |
| 1-Me-pyrazol-4-yl | NH-(1-Me-piperidin-4-yl) |
| 1-Me-pyrazol-4-yl | NMeCH₂CH₂CH₂NMe₂ |
| 1-Me-pyrazol-4-yl | azetidin-1-yl |
| 1-Me-pyrazol-4-yl | NHCH₂CH₂-pyrrolidin-1-yl |
| 1-Me-pyrazol-4-yl | NMe₂ |
| 1-Me-pyrazol-4-yl | 4-(Me₂N)-piperidin-1-yl |
| 1-Me-pyrazol-4-yl | NHCH₂CH₂CH₂NMe₂ |
| 1-Me-pyrazol-4-yl | NHCH₂CH₂NMe₂ |
| 1-Me-pyrazol-4-yl | NHCH₂CH₂OMe |
| 1-Me-pyrazol-4-yl | NMe-(1-Me-piperidin-4-yl) |
| 1-Me-pyrazol-4-yl | NMeCH₂-(1-Me-piperidin-2-yl) |
| 1-Me-pyrazol-4-yl | NMeCH₂-(1-Me-piperidin-3-yl) |
| 1-Me-pyrazol-4-yl | 4-(Me₂NCH₂CH₂CH₂)-piperidin-1-yl |
| 1-Me-pyrazol-4-yl | NHCH₂-(1-Me-pyrrolidin-3-yl |
| 1-Me-pyrazol-4-yl | 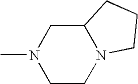 |
| 1-Me-pyrazol-4-yl | 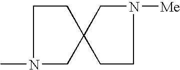 |

Compounds of formulae IA and IB may be prepared by the methods disclosed in the aforementioned WO 01/02369, WO 03/024969, US 2004/0242559 and WO 00/6986 or simple adaptations thereof. In a typical route to a compound of formula IA, an indazole derivative (1) is coupled with an indole derivative (2), followed by removal of the protecting groups:

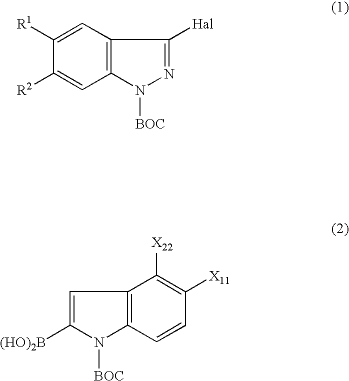

where Hal represents Cl, Br or I (preferably Br or I), BOC represents t-butoxycarbonyl, X11 and X22 represent X1 and X2 respectively or synthetic precursors thereof, and R¹, R², X1 and X2 have the same meanings as before. The coupling takes place in the presence of a Pd(0) catalyst such as Pd(PPh₃)₄ and a base such as sodium carbonate in an ethereal solvent (e.g. aqueous dimethoxyethane) at reflux. The BOC protecting groups are removed by treatment with trifluoroacetic acid.

Compounds (1) are obtained by halogenation of the corresponding indazoles (e.g. by treatment with iodine and KOH in DMF), followed by treatment with (BOC)₂O and dimethylaminopyridine.

Compounds of formula IB are obtainable by the analogous route starting with the relevant tetrahydroindazoles, which may be obtained by treatment of the appropriate cyclohexanones with NaOEt and ethyl formate followed by hydrazine hydrate.

In the indole derivatives (2), X11 or X22 advantageously represents CH₂OSiR₃ where each R independently represents C₁₋₄alkyl (e.g. n-butyl). The preparation of such compounds is disclosed in WO 01/29025. Subsequent to coupling with the indazole derivative, cleavage of the silyl ether (e.g. by treatment with HF/Et₃N) provides compounds in which X1 or X2 is CH₂OH. These may be alkylated by standard methods to provide other compounds in which X1 or X2 is CH₂OR³. Alternatively, the hydroxymethyl group may be oxidised (e.g. using Dess-Martin reagent) to the aldehyde, which may then be reacted with R³R⁴NH and sodium triacetoxyborohydride to provide compounds in which X1 or X2 is CH₂NR³R⁴.

Compounds of formula IA in which R¹ or R² represents Ar are conveniently obtained by coupling of the corresponding compounds in which R¹ or R² is Cl, Br or I with Ar—B(OH)₂. The reaction may be carried out in similar manner to the coupling of (1) with (2).

Where they are not themselves commercially available, the starting materials and reagents described above may be obtained from commercially available precursors by means of well known synthetic procedures and/or the methods disclosed in the Examples section herein.

Where the above-described processes for the preparation of the compounds of use in the invention give rise to mixtures of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques such as preparative HPLC, or the formation of diastereomeric pairs by salt formation with an optically active acid, such as di-p-toluoyl-D-tartaric acid and/or di-p-toluoyl-L-tartaric acid, followed by fractional crystallization and regeneration of the free base. The compounds may also be resolved by formation of diastereomeric esters or amides, followed by chromatographic separation and removal of the chiral auxiliary.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The compounds of formula IA or IB are suitably administered to patients in the form a pharmaceutical composition comprising the active ingredient (i.e. the compound of formula IA or IB or pharmaceutically acceptable salt or hydrate thereof) and a pharmaceutically acceptable carrier.

Preferably these compositions are in unit dosage forms such as tablets, pills, capsules, powders, granules, sterile parenteral solutions or suspensions, metered aerosol or liquid sprays, drops, ampoules, transdermal patches, auto-injector devices or suppositories; for oral, parenteral, intranasal, sublingual or rectal administration, or for administration by inhalation or insufflation. The principal active ingredient typically is mixed with a pharmaceutical carrier, e.g. conventional tableting ingredients such as corn starch, lactose, sucrose, sorbitol, talc, stearic acid, magnesium stearate and dicalcium phosphate, or gums, dispersing agents, suspending agents or surfactants such as sorbitan monooleate and polyethylene glycol, and other pharmaceutical diluents, e.g. water, to form a homogeneous preformulation composition containing a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This preformulation composition is then subdivided into unit dosage forms of the type described above containing from 0.1 to about 500 mg of the active ingredient of the present invention. Typical unit dosage forms contain from 1 to 100 mg, for example 1, 2, 5, 10, 25, 50 or 100 mg, of the active ingredient. Tablets or pills of the composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions useful in the present invention may be incorporated for administration orally or by injection include aqueous solutions, liquid- or gel-filled capsules, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, poly(ethylene glycol), poly(vinylpyrrolidone) or gelatin.

In one embodiment of the invention, the compound of formula IA or IB is administered to a patient suffering from AD, FTDP-17, Pick's disease or frontotemporal dementia, preferably AD.

In an alternative embodiment of the invention, the compound of formula IA or IB is administered to a patient suffering from mild cognitive impairment or age-related cognitive decline. A favourable outcome of such treatment is prevention or delay of the onset of AD. Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). As used herein, “age-related cognitive decline” implies a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition MCI, the degree of memory impairment is outside the range considered normal for the age of the patient but AD is not present. The differential diagnosis of MCI and mild AD is described by Petersen et al, Arch. Neurol., 56 (1999), 303-8. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Within this embodiment, the compound of formula IA or IB is advantageously administered to patients who suffer impaired memory function but do not exhibit symptoms of dementia. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; and adult-onset diabetes mellitus.

In a particular embodiment of the invention, the compound of formula IA or IB is administered to a patient suffering from age-related cognitive decline or MCI who additionally possesses one or more risk factors for developing AD selected from: a family history of the disease; a genetic predisposition to the disease; elevated serum cholesterol; adult-onset diabetes mellitus; elevated baseline hippocampal volume; elevated CSF levels of total tau; elevated CSF levels of phospho-tau; and lowered CSF levels of Aβ(1-42).

A genetic predisposition (especially towards early onset AD) can arise from point mutations in one or more of a number of genes, including the AβP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment in accordance with the invention, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assocn. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J. Psychiatry, 141 (1984), 1356-64).

For treating or preventing Alzheimer's disease, a suitable dosage level is about 0.01 to 250 mg/kg per day, preferably about 0.01 to 100 mg/kg per day, and more preferably about 0.05 to 50 mg/kg of body weight per day, of the active compound. The compounds may be administered on a regimen of 1 to 4 times per day. In some cases, however, a dosage outside these limits may be used.

The compound of formula IA or IB optionally may be administered in combination with one or more additional compounds known to be useful in the treatment or prevention of AD or the symptoms thereof. Such additional compounds thus include cognition-enhancing drugs such as acetylcholinesterase inhibitors (e.g. donepezil and galanthamine), NMDA antagonists (e.g. memantine) or PDE4 inhibitors (e.g. Ariflo™ and the classes of compounds disclosed in WO 03/018579, WO 01/46151, WO 02/074726 and WO 02/098878). Such additional compounds also include cholesterol-lowering drugs such as the statins, e.g. simvastatin. Such additional compounds similarly include compounds known to modify the production or processing of Aβ in the brain (“amyloid modifiers”), such as compounds which modulate the secretion of Aβ (including γ-secretase inhibitors, γ-secretase modulators and β-secretase inhibitors), compounds which inhibit the aggregation of Aβ, and antibodies which selectively bind to Aβ. Such additional compounds further include growth hormone secretagogues, e.g. as described in WO 2004/080459.

In this embodiment of the invention, the amyloid modifier may be a compound which inhibits the secretion of Aβ, for example an inhibitor of γ-secretase (such as those disclosed in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, WO 2004/089911, WO 02/081435, WO 02/081433, WO 03/018543, WO 2004/031137, WO 2004/031139, WO 2004/031138, WO 2004/101538, WO 2004/101539 and WO 02/47671), or a β-secretase inhibitor (such as those disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820), or any other compound which inhibits the formation or release of Aβ including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621, and also including GSK-3 inhibitors, particularly GSK-3α inhibitors, such as lithium, as disclosed in Phiel et al, Nature, 423 (2003), 435-9.

Alternatively, the amyloid modifier may be a compound which modulates the action of γ-secretase so as to selectively attenuate the production of Aβ(1-42). Compounds reported to show this effect include certain non-steroidal antiinflammatory drugs (NSAIDs) and their analogues (see WO 01/78721 and US 2002/0128319 and Weggen et al Nature, 414 (2001) 212-16; Morihara et al, J. Neurochem., 83 (2002), 1009-12; and Takahashi et al, J. Biol. Chem., 278 (2003), 18644-70), and compounds which modulate the activity of PPARα and/or PPARδ (WO 02/100836). Further examples of γ-secretase modulators are disclosed in WO 2005/054193, WO 2005/013985, WO 2005/108362, WO 2006/008558 and WO 2006/043064.

Alternatively, the amyloid modifier may be a compound which inhibits the aggregation of Aβ or otherwise attenuates is neurotoxicicity. Suitable examples include chelating agents such as clioquinol (Gouras and Beal, Neuron, 30 (2001), 641-2) and the compounds disclosed in WO 99/16741, in particular that known as DP-109 (Kalendarev et al, J. Pharm. Biomed. Anal., 24 (2001), 967-75). Other inhibitors of Aβ aggregation suitable for use in the invention include the compounds disclosed in WO 96/28471, WO 98/08868 and WO 00/052048, including the compound known as Apan™ (Praecis); WO 00/064420, WO 03/017994, WO 99/59571 (in particular 3-aminopropane-1-sulfonic acid, also known as tramiprosate or Alzhemed™); WO 00/149281 and the compositions known as PTI-777 and PTI-00703 (ProteoTech); WO 96/39834, WO 01/83425, WO 01/55093, WO 00/76988, WO 00/76987, WO 00/76969, WO 00/76489, WO 97/26919, WO 97/16194, and WO 97/16191. Further examples include phytic acid derivatives as disclosed in U.S. Pat. No. 4,847,082 and inositol derivatives as taught in US 2004/0204387.

Alternatively, the amyloid modifier may be an antibody which binds selectively to Aβ. Said antibody may be polyclonal or monoclonal, but is preferably monoclonal, and is preferably human or humanized. Preferably, the antibody is capable of sequestering soluble Aβ from biological fluids, as described in WO 03/016466, WO 03/016467, WO 03/015691 and WO 01/62801. Suitable antibodies include humanized antibody 266 (described in WO 01/62801) and the modified version thereof described in WO 03/016466. Suitable antibodies also include those specific to Aβ-derived diffusible ligands (ADDLS), as disclosed in WO 2004/031400.

As used herein, the expression “in combination with” requires that therapeutically effective amounts of both the compound of formula IA or IB and the additional compound are administered to the subject, but places no restriction on the manner in which this is achieved. Thus, the two species may be combined in a single dosage form for simultaneous administration to the subject, or may be provided in separate dosage forms for simultaneous or sequential administration to the subject. Sequential administration may be close in time or remote in time, e.g. one species administered in the morning and the other in the evening. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. When the additional compound is an antibody, it will typically be administered parenterally and separately from the compound of formula IA or IB.

### EXAMPLES

### Assay for MARK Inhibition

MARK3 activity was assayed in vitro using a Cdc25C biotinylated peptide substrate (Cell Signalling Technologies). The phosphopeptide product was quantitated using a Homogenous Time-Resolved Fluorescence (HTRF) assay system (Park et al., 1999, Anal. Biochem. 269:94-104). The reaction mixture contained 50 mM HEPES/Tris-HCl, pH 7.4; 10 mM NaCl, 5 mM MgCl₂, 0.2 mM NaVO₄, 5 mM β-glycerol phosphate, 0.1% Tween-20, 2 mM dithiothreitol, 0.1% BSA, 10 μM ATP, 1 μM peptide substrate, and 10 nM recombinant MARK3 enzyme (University of Dundee) in a final volume of 12 μl. The buffer additionally contained protease inhibitor cocktail (Roche EDTA-free, 1 tab per 50 ml). The kinase reaction was incubated for 2 hours at 25° C., and then terminated with 3 μl Stop/Detection Buffer (50 mM HEPES, pH 7.0, 16.6 mM EDTA, 0.5M KF, 0.1% Tween-20, 0.1% BSA, 2 μg/ml SLX^{ent} 665 (CISBIO), and 2 μg/ml Eu³⁺ cryptate label antibody (CISBIO)). The reaction was allowed to equilibrate overnight at 0° C., and relative fluorescent units were read on an HTRF enabled plate reader (e.g. TECAN GENios Pro).

Inhibitor compounds were assayed in the reaction described above to determine compound IC50s. Aliquots of compound dissolved in DMSO were added to the reaction wells in a third-log dilution series covering a range of 1 nM to 10 μM. Relative phospho substrate formation, read as HTRF fluorescence units, was measured over the range of compound concentrations and a titration curve generated.

The compounds disclosed herein gave an IC50 of less than 2 μM, typically less than 0.5 μM, and in preferred cases less than 50 nM in the above assay.

### SYNTHESIS EXAMPLES

Representative synthetic methods for the compounds suitable for use in the invention are described below.

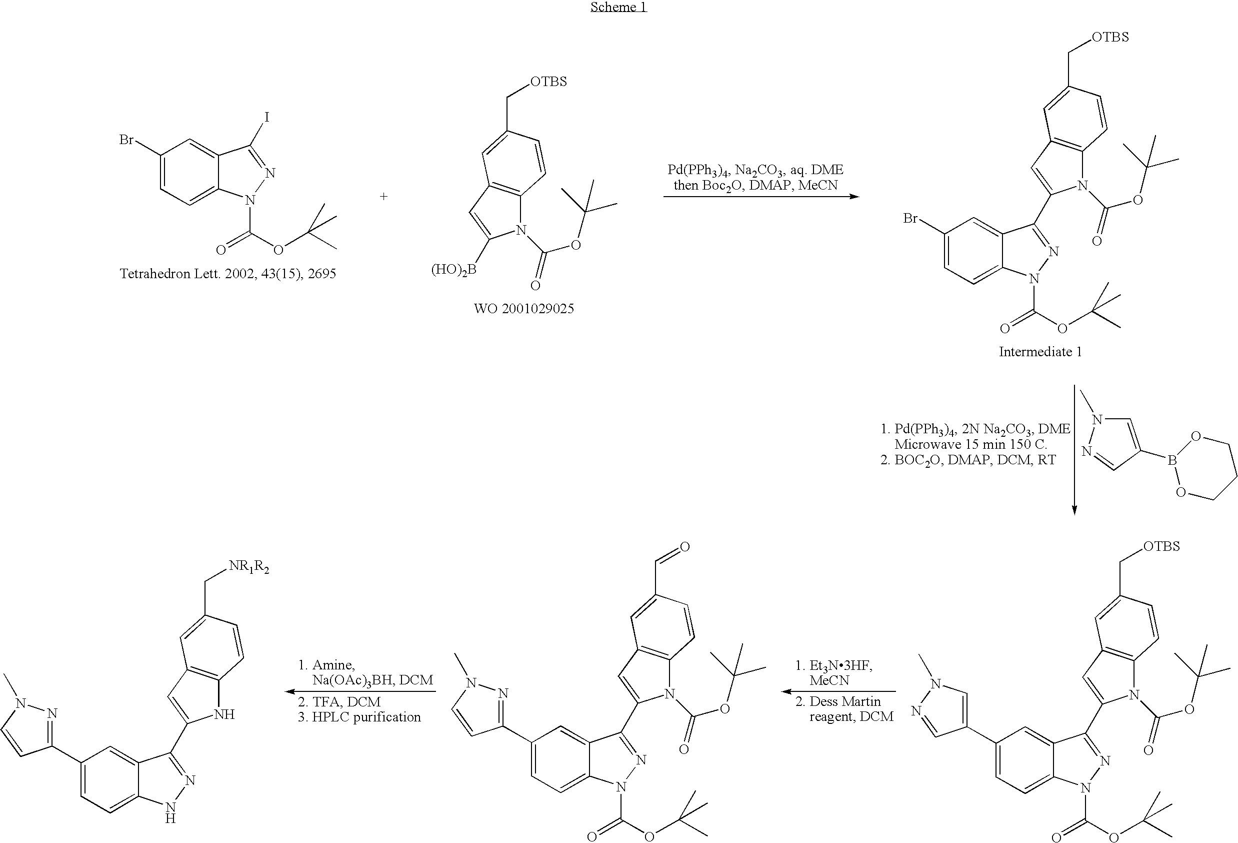

### Scheme 1—Step 1:

[1-(tert-Butoxycarbonyl)-5-({[tert-butyl(dimethyl)silyl]oxy}methyl)-1H-indol-2-yl]boronic acid (10 g, 23.6 mmol, prepared as in Tetrahedron Lett. 2002, 43(15), 2695) and tert-butyl 5-bromo-3-iodo-1H-indazole-1-carboxylate (7 g, 17.3 mmol, prepared as in WO2001029025) were dissolved in DME (126 ml). 2N Na₂CO₃ (49 ml) was added and the reaction mixture was stirred for 0.5 h. Freshly prepared Pd(PPh₃)₄ (80 mg, 0.69 mmol) was then added and the mixture heated overnight at 85° C. On cooling, the reaction was diluted with brine and extracted with EtOAc (×3), dried (MgSO₄) and evaporated in vacuo. The crude material was dissolved in acetonitrile (100 ml) and (Boc)₂O (5.4 g, 24.5 mmol) and DMAP (3.0 g, 24.5 mmol) was added. Reaction stirred for 2 h at RT. Solvent removed in vacuo. Reaction mixture was then dissolved in EtOAc and washed with 0.1N HCl and brine, dried and solvent evaporated. Product purified by flash column chromatography (silica gel eluant 10% EtOAc/Hexane to afford Intermediate 1 (11 g).

### Scheme 1—Step 2:

To a solution of 4-(1,3,2-dioxaborinan-2-yl)-1-methyl-1H-pyrazole (640 mg, 3.8 mmol) and Intermediate 1 (500 mg, 0.76 mmol) in DME (5 ml) in a microwave vial was added 2N sodium carbonate (5 ml) and Pd(PPh₃)₄ (150 mg, 0.123 mmol). The reaction was sealed and the mixture heated to 150° C. for 15 minutes in a microwave reactor. On cooling, the reaction was diluted with sodium bicarbonate solution and extracted with EtOAc (×3), dried (MgSO₄) and evaporated in vacuo. The product was purified by flash column chromatography (silica gel eluent 30:70 EtOAc:Hexane). (A variety of boronic acids or esters may be used in this step in place of 4-(1,3,2-dioxaborinan-2-yl)-1-methyl-1H-pyrazole).

### Scheme 1—Step 3:

The silyl ether from the foregoing step (4.6 g, 7.0 mmol) was dissolved in acetonitrile (100 ml) and triethylamine trihydrofluoride (3.6 g, 22 mmol) was added. The reaction was stirred at RT for 3 h. Aqueous sodium bicarbonate solution was added and the product was extracted with EtOAc (3×), dried (Na₂SO₄) and solvent removed in vacuo to afford the desired alcohol which was used without further purification.

### Scheme 1—Step 4:

The alcohol from the foregoing step (750 mg, 1.38 mmol) was dissolved in DCM (15 ml), Dess-Martin Periodinane (879 mg, 2.07 mmol) then added and the mixture stirred at RT for 3 h. The reaction was diluted with further DCM (20 ml) and washed with solutions of sodium thiosulphate, sodium bicarbonate (×3) and brine (×1) then dried (Na₂SO₄) and evaporated in vacuo to afford the desired aldehyde which was used without further purification.

### Scheme 1—Step 5:

The aldehyde from the foregoing step (150 mg, 0.28 mmol) was dissolved in DCM (5 ml) and the appropriate amine (0.5 mmol) was then added. The reaction was stirred at RT for 1 h, allowing the formation of the imine, before the addition of sodium triacetoxyborohydride (240 mg, 1.11 mmol). Reaction stirred at RT overnight then water and DCM were added, the layers separated and the organic layer evaporated. Product was used without further purification.

### Scheme 1—Step 6:

The crude residue from the foregoing step was dissolved in DCM/TFA (1 ml), stirred 1 h and evaporated. The product was purified by HPLC.

### Scheme 2

Scheme 2 involves the same procedures as Scheme 1, but carried out in a different sequence. Thus, Intermediate 1 (prepared as in Scheme 1) was reacted under the condition of Scheme 1, steps 3 and 4 to afford an aldehyde which was subjected to reductive amination with the appropriate amine (such as morpholine, piperidine, Boc(1,4-diaminocyclohexane etc.) under the conditions of Scheme 1, step 5. The resulting intermediate was subjected to Suzuki cross-coupling conditions using the procedure of Scheme 1, step 2 using the appropriate boronic acid/ester, then deprotected and purified using the procedure of Scheme 1, step 6.

By varying the identities of the amine, the boronic acid or ester, and/or the indazole starting material, a wide variety of 5- and 6-substituted indazole analogs were obtained using the procedures outlined above, as summarised in the following table:

| | | | Synthesis |
|---|---|---|---|
| Example | Structure | ms [MH+] | Scheme |
| 1 | 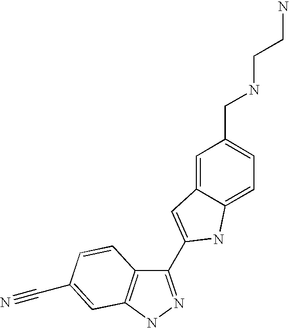 | 331 | 1 |
| 2 | 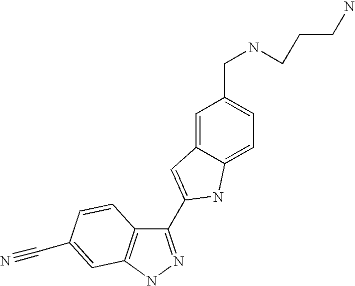 | 345 | 1 |
| 3 | 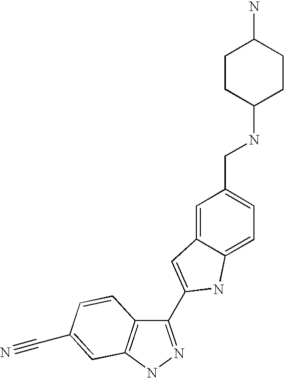 | 385 | 1 |
| 4 | 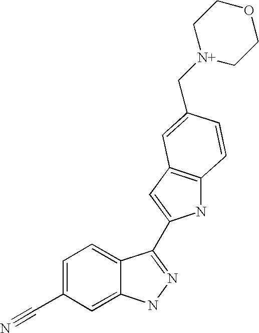 | 358 | 1 |
| 5 | 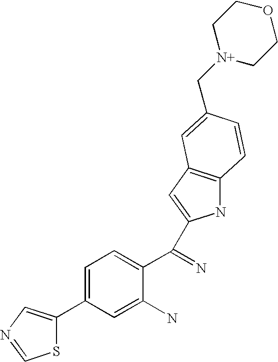 | 416 | 2 |
| 6 | 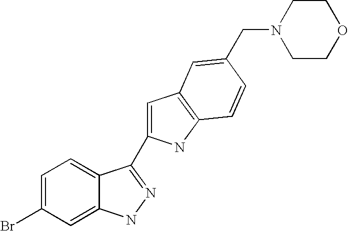 | 412 | 2 |
| 7 | 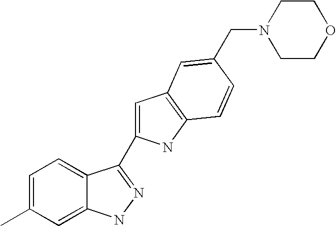 | 347 | 2 |
| 8 | 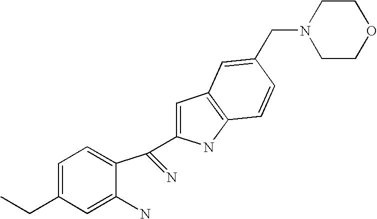 | 361 | 2 |
| 9 | 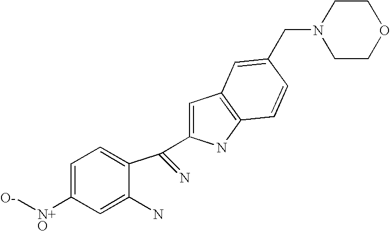 | 378 | 2 |
| 10 | 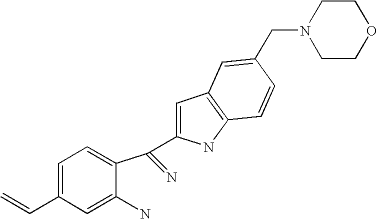 | 359 | 2 |
| 11 | 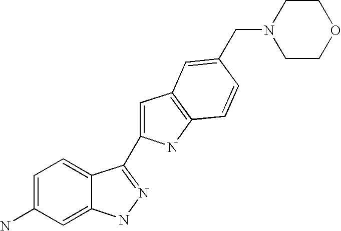 | 348 | 2 |
| 12 | 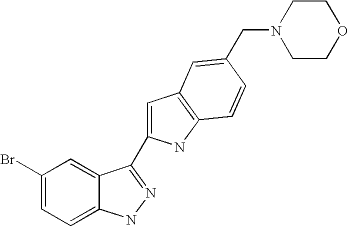 | 412 | 2 |
| 13 | 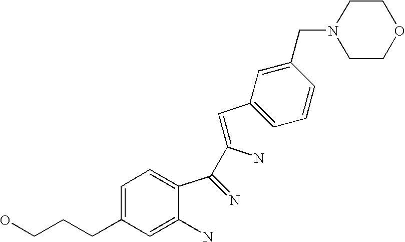 | 391 | 2 |
| 14 | 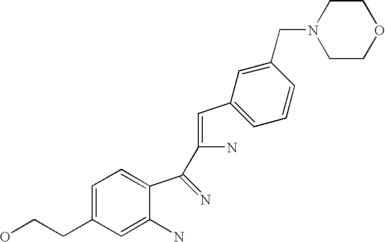 | 377 | 2 |
| 15 | 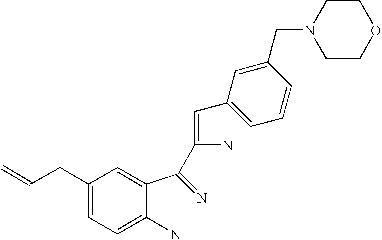 | 373 | 2 |
| 16 | 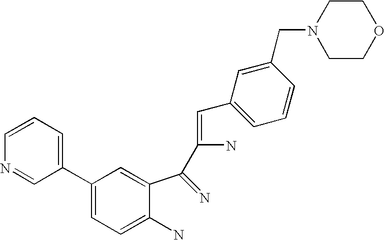 | 410 | 2 |
| 17 | 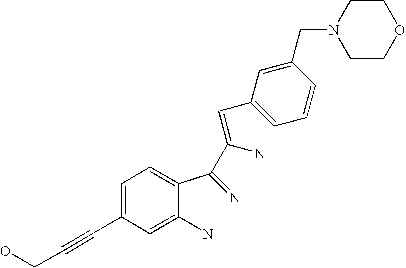 | 387 | 2 |
| 18 | 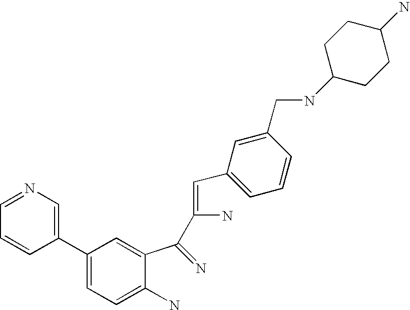 | 437 | 1 |
| 19 | 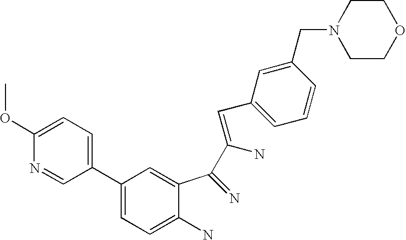 | 440 | 2 |
| 20 | 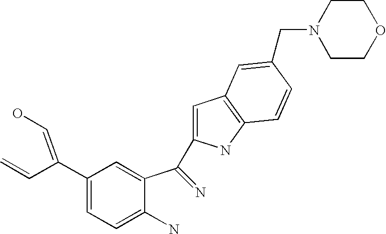 | 399 | 2 |
| 21 | 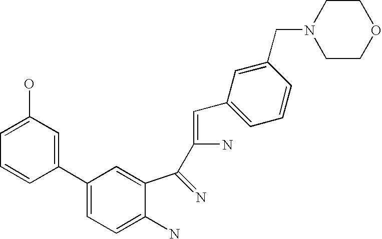 | 425 | 2 |
| 22 | 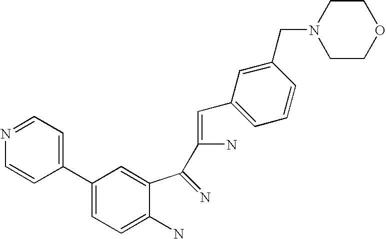 | 410 | 2 |
| 23 | 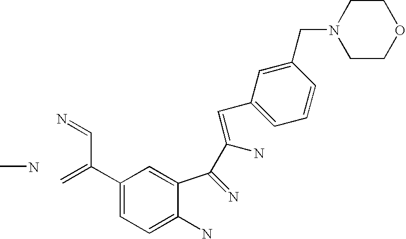 | 413 | 2 |
| 24 | 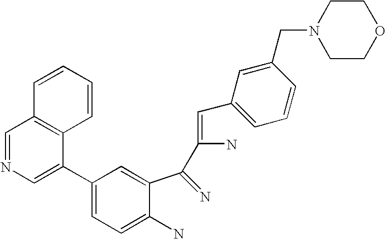 | 460 | 2 |
| 25 | 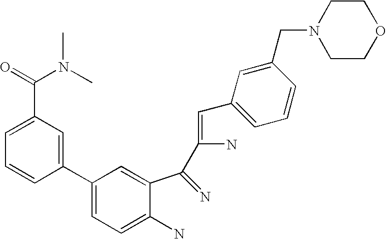 | 480 | 2 |
| 26 | 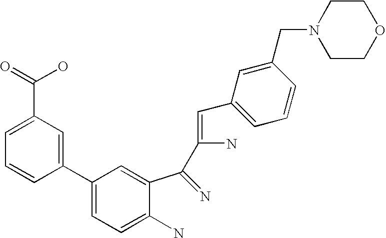 | 453 | 2 |
| 27 | 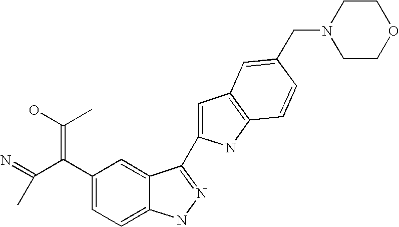 | 428 | 2 |
| 28 | 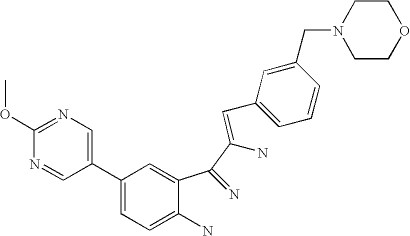 | 441 | 2 |
| 29 | 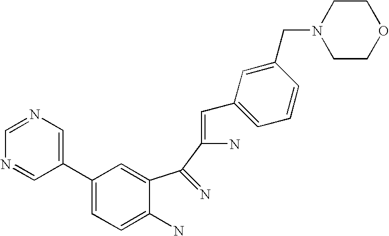 | 411 | 2 |
| 30 | 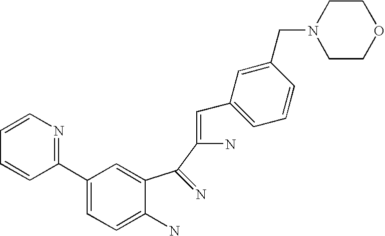 | 410 | 2 |
| 31 | 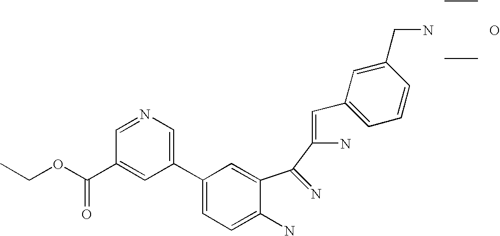 | 482 | 2 |
| 32 | 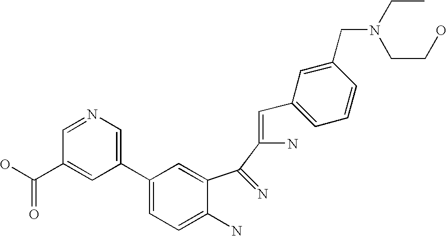 | 454 | 2 |
| 33 | 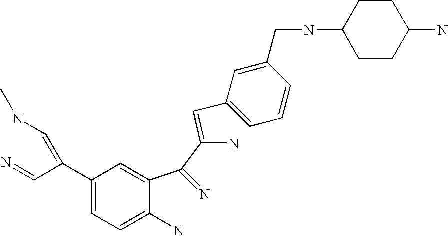 | 440 | 1 |
| 34 | 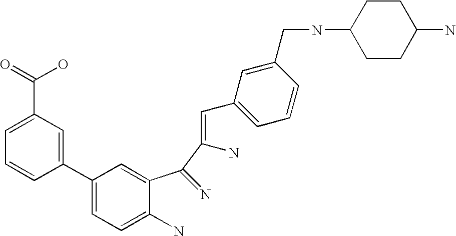 | 480 | 2 |
| 35 | 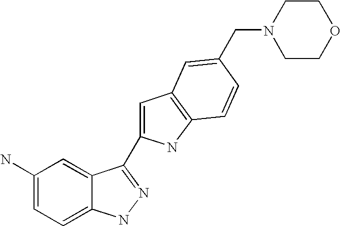 | 348 | 2 |
| 36 | 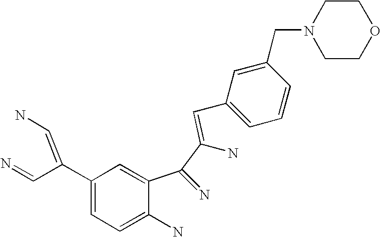 | 399 | 2 |
| 37 | 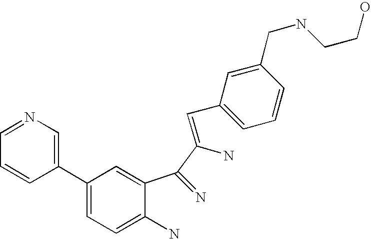 | 384 | 1 |
| 38 | 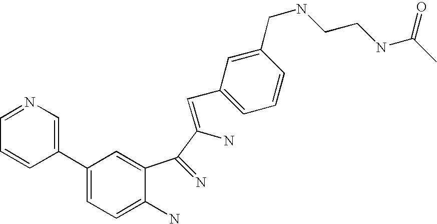 | 425 | 1 |
| 39 | 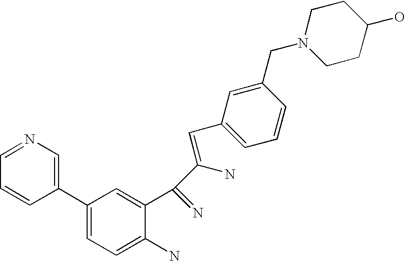 | 424 | 1 |
| 40 | 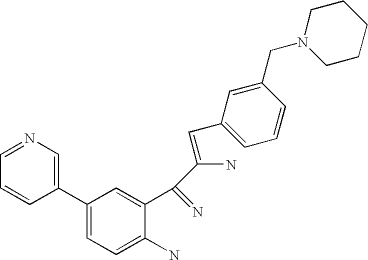 | 408 | 1 |
| 41 | 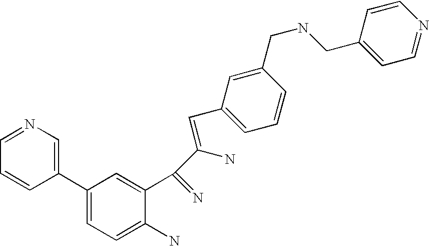 | 431 | 1 |
| 42 | 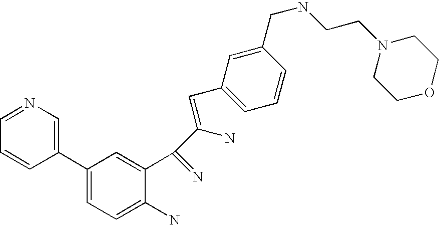 | 453 | 1 |
| 43 | 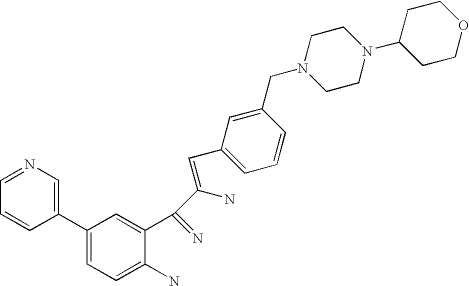 | 493 | 1 |
| 44 | 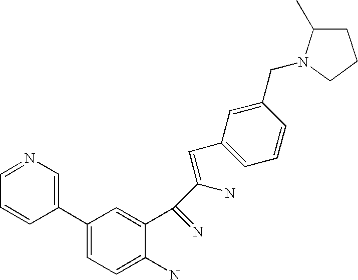 | 408 | 1 |
| 45 | 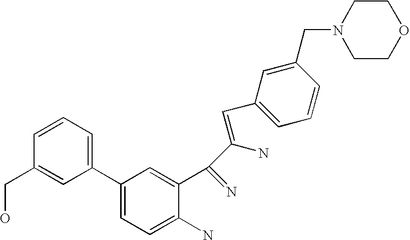 | 439 | 2 |
| 46 | 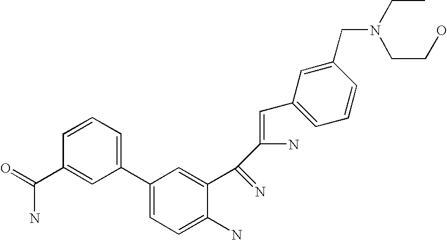 | 452 | 2 |
| 47 | 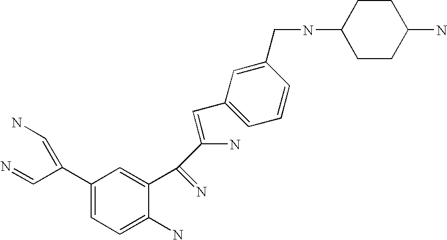 | 426 | 1 |
| 48 | 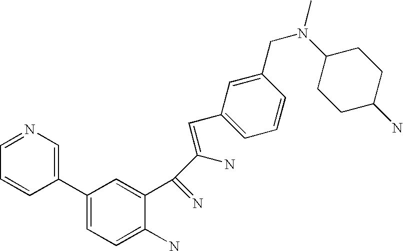 | 451 | 1 |
| 49 | 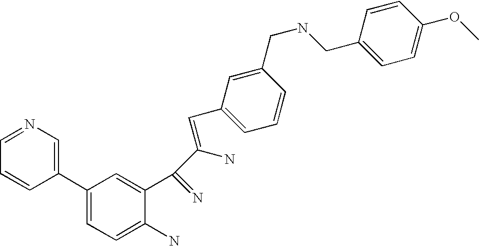 | 460 | 1 |
| 50 | 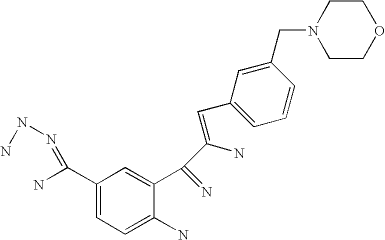 | 401 | 2 |
| 51 | 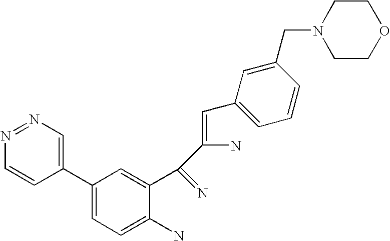 | 411 | 2 |
| 52 | 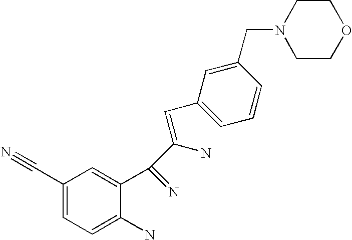 | 358 | 2 |
| 53 | 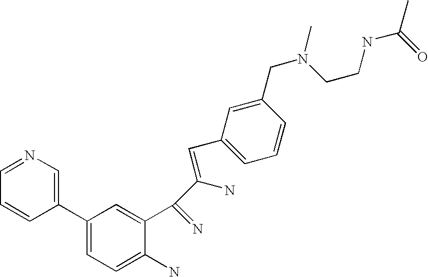 | 439 | 1 |
| 54 | 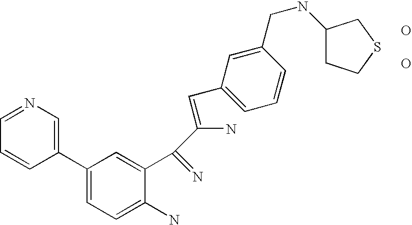 | 458 | 1 |
| 55 | 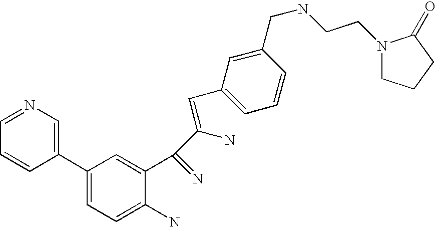 | 451 | 1 |
| 56 | 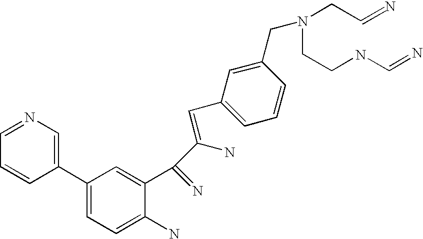 | 447 | 1 |
| 57 | 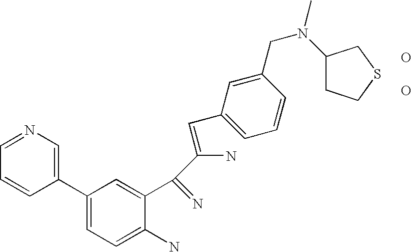 | 472 | 1 |
| 58 | 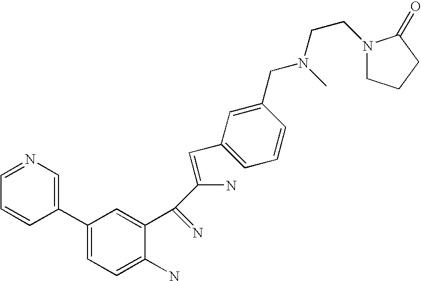 | 465 | 1 |
| 59 | 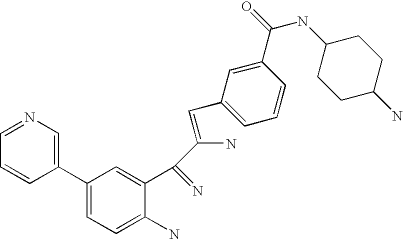 | 451 | 1 |
| 60 | 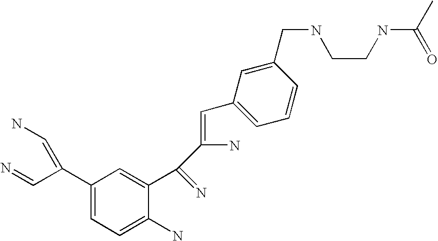 | 414 | 1 |
| 61 | 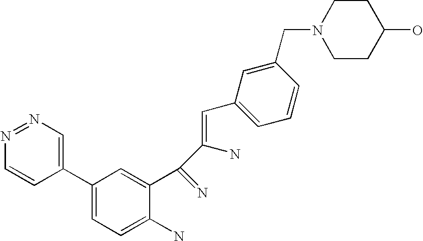 | 425 | 1 |
| 62 | 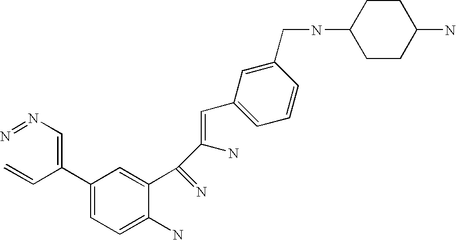 | 438 | 1 |
| 63 | 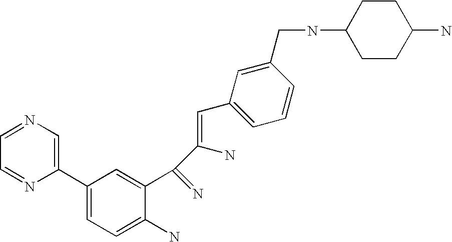 | 438 | 2 |
| 64 | 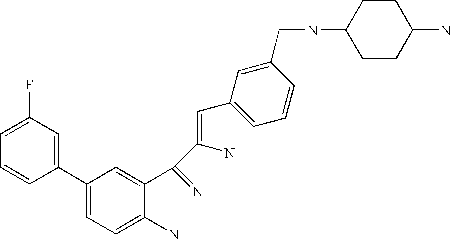 | 454 | 2 |
| 65 | 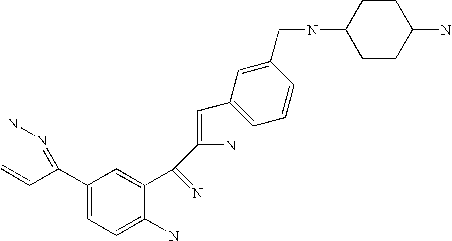 | 426 | 2 |
| 66 | 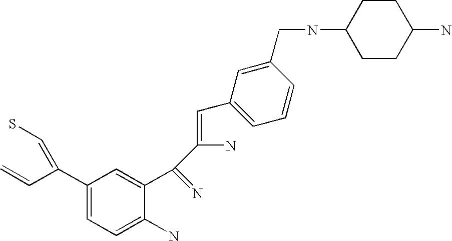 | 442 | 2 |
| 67 | 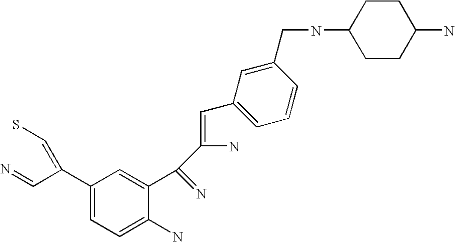 | 443 | 2 |
| 68 | 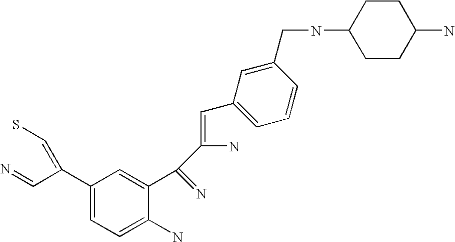 | 443 | 2 |
| 69 | 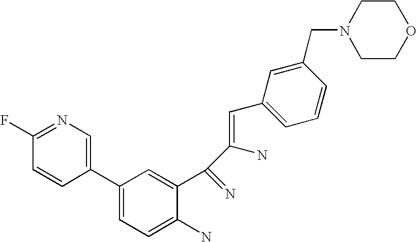 | 428 | 2 |
| 70 | 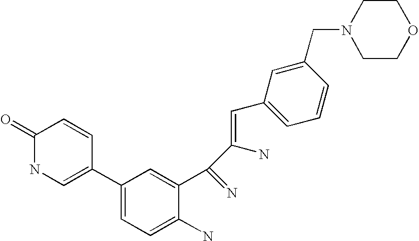 | 426 | 2 |
| 71 | 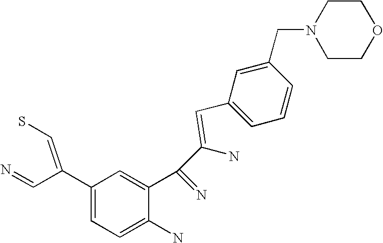 | 416 | 2 |
| 72 | 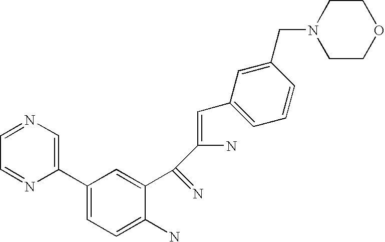 | 411 | 2 |
| 73 | 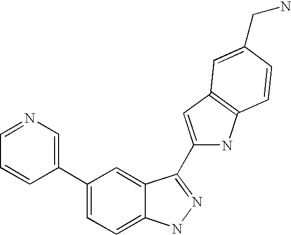 | 340 | 1 |
| 74 | 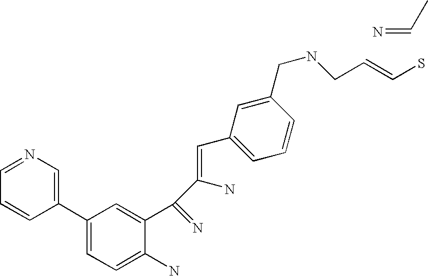 | 451 | 1 |
| 75 | 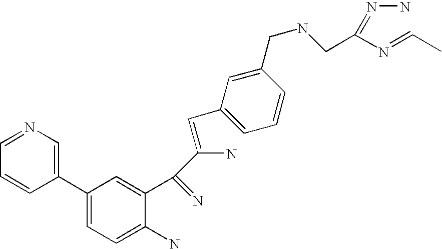 | 435 | 1 |
| 76 | 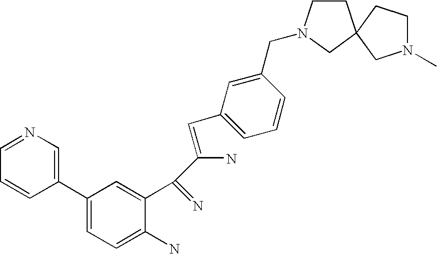 | 463 | 1 |
| 77 | 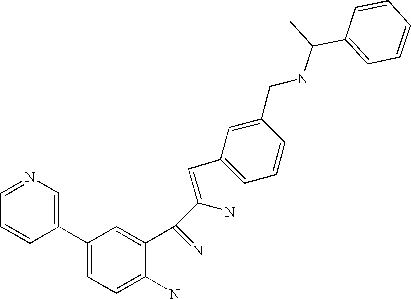 | 444 | 1 |
| 78 | 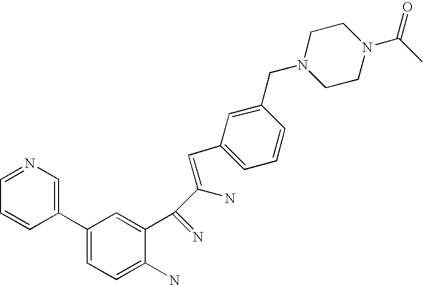 | 451 | 1 |
| 79 | 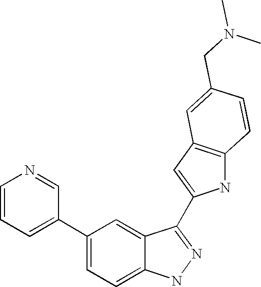 | 368 | 1 |
| 80 | 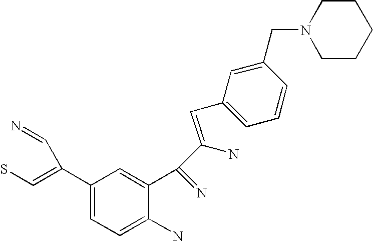 | 414 | 1 |
| 81 | 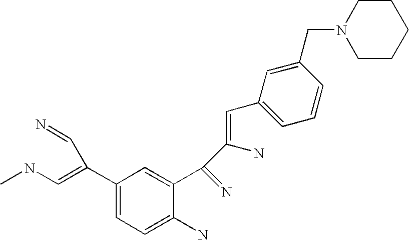 | 411 | 2 |
| 82 | 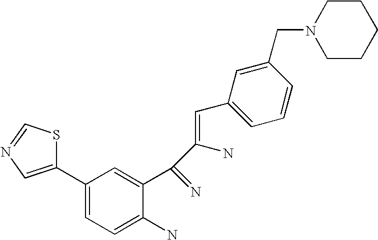 | 414 | 2 |
| 83 | 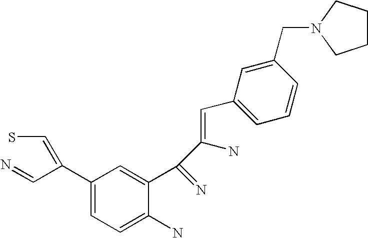 | 400 | 1 |
| 84 | 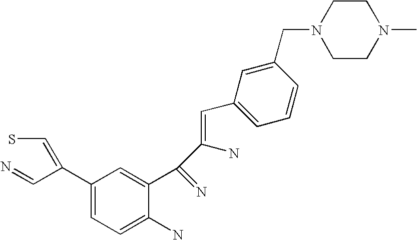 | 429 | 1 |
| 85 | 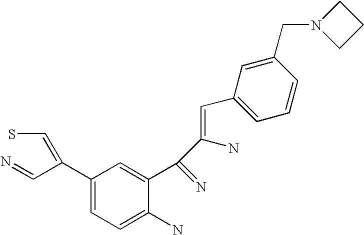 | 386 | 1 |
| 86 | 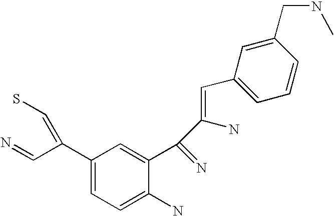 | 360 | 1 |
| 87 | 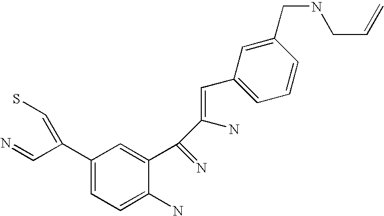 | 386 | 1 |
| 88 | 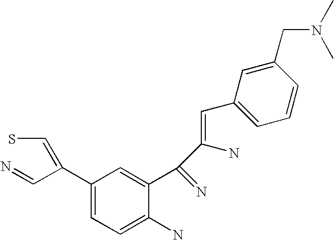 | 374 | 1 |
| 89 | 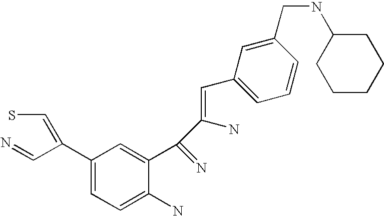 | 428 | 1 |
| 90 | 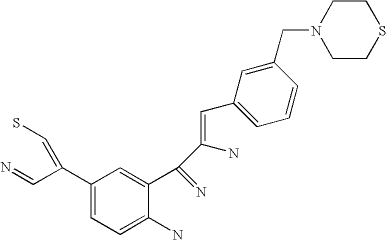 | 432 | 1 |
| 91 | 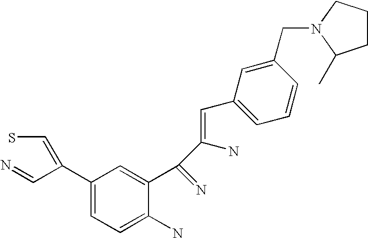 | 414 | 1 |
| 92 | 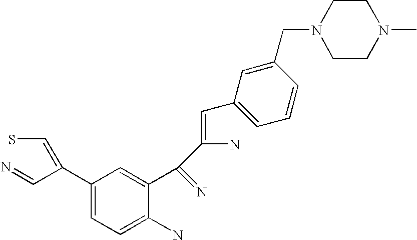 | 443 | 1 |
| 93 | 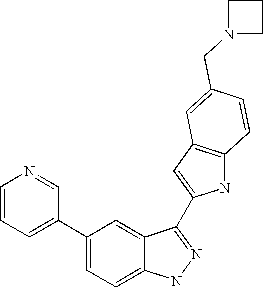 | 380 | 1 |
| 94 | 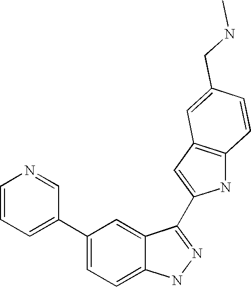 | 354 | 1 |
| 95 | 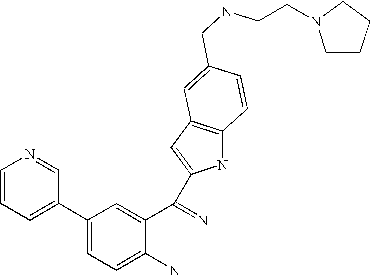 | 437 | 1 |
| 96 | 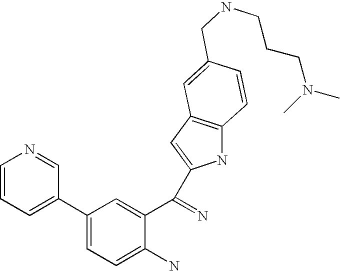 | 425 | 1 |
| 97 | 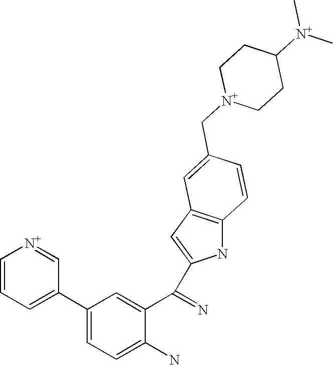 | 451 | 1 |
| 98 | 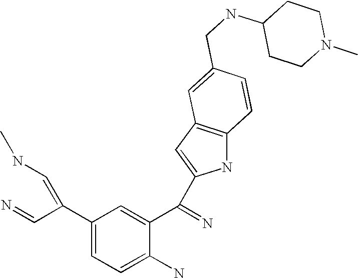 | 440 | 1 |
| 99 | 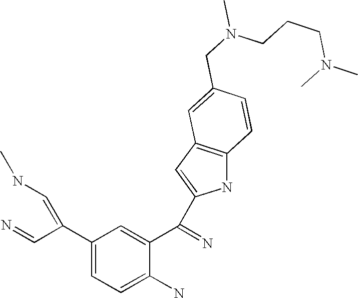 | 442 | 1 |
| 100 | 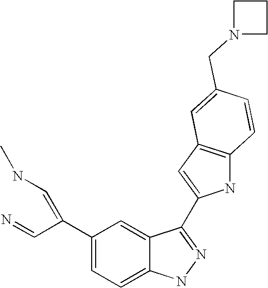 | 383 | 1 |
| 101 | 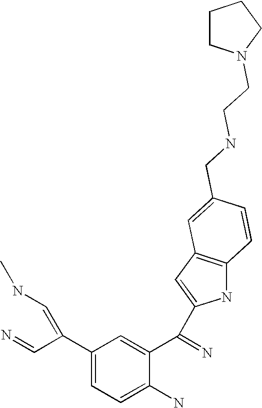 | 440 | 1 |
| 102 | 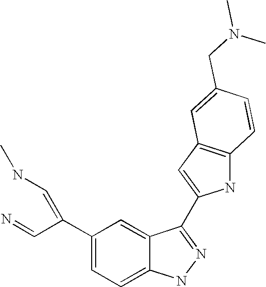 | 371 | 1 |
| 103 | 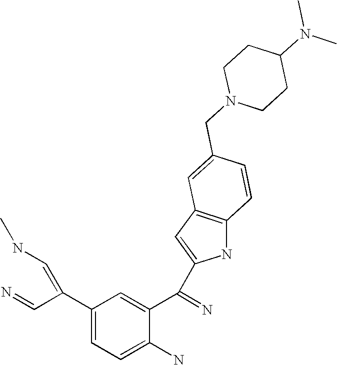 | 454 | 1 |
| 104 | 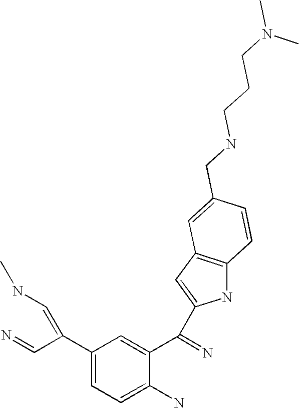 | 428 | 1 |
| 105 | 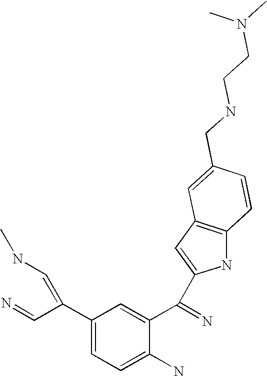 | 414 | 1 |
| 106 | 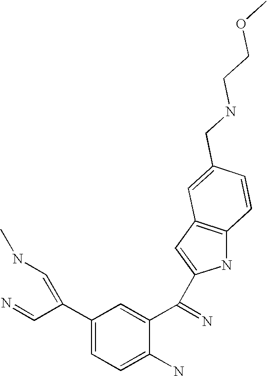 | 401 | 1 |
| 107 | 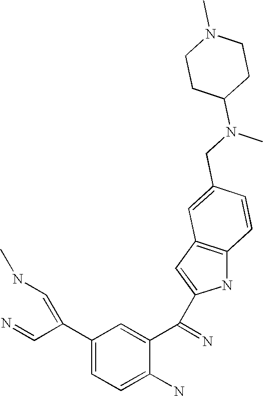 | 454 | 1 |
| 108 | 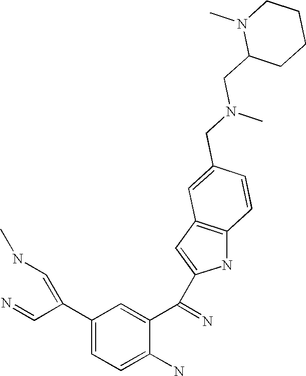 | 468 | 1 |
| 109 | 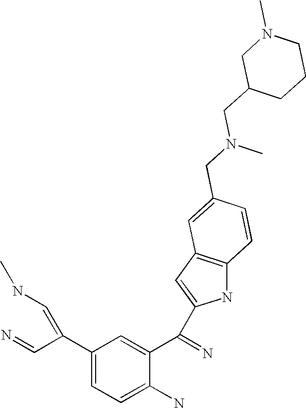 | 468 | 1 |
| 110 | 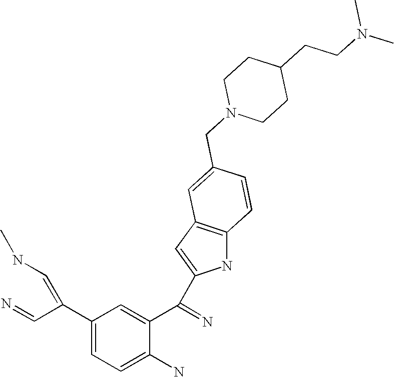 | 482 | 1 |
| 111 | 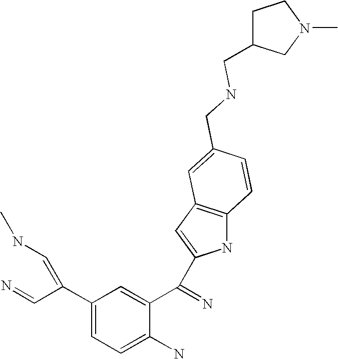 | 440 | 1 |
| 112 | 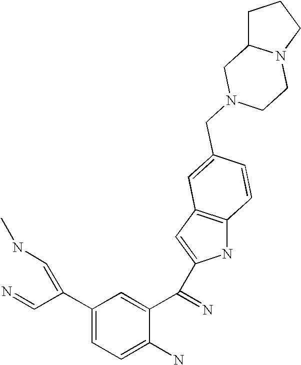 | 452 | 1 |
| 113 | 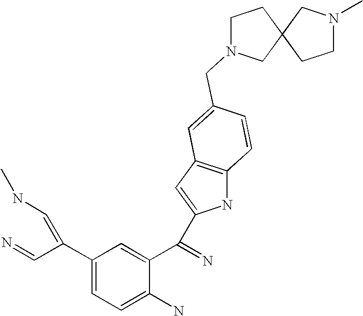 | 466 | 1 |

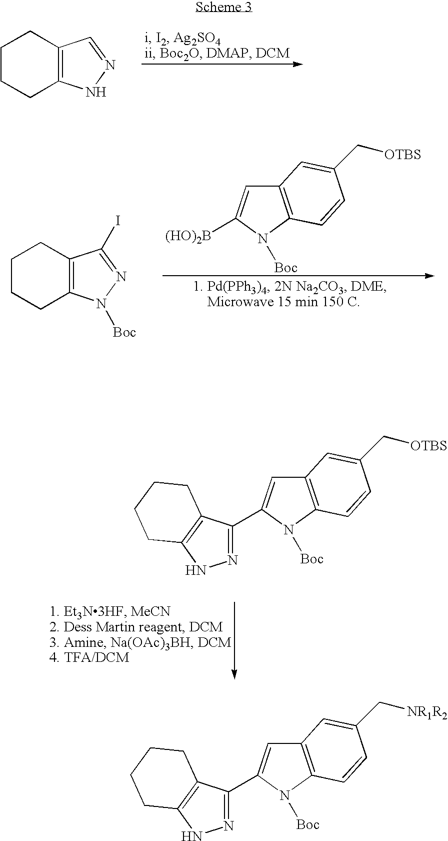

### Scheme 3—Step 1

A solution of 4,5,6,7-tetrahydroindazole (5.1 g), silver sulphate (17 g) and iodine (14 g) in ethanol (100 ml) was stirred at room temperature for 18 hours then filtered and evaporated. The residue was triturated with DCM then columned in 1-4% MeOH:DCM to afford the desired iodotetrahydroindazole (4 g).

### Scheme 3—Step 2

To the iodotetrahydroindazole from the foregoing step (0.85 g) in MECN (5 ml) was added Boc₂O (1.1 eq.) and DMAP (1 eq.). The mixture was stirred for 5 minutes then evaporated and taken up in EtOAc/water. The layers were separated and the organic layer washed with 1N HCl, water and brine then dried (MgSO₄) and evaporated. Used without further purification.

### Scheme 3—Step 3

[1-(tert-Butoxycarbonyl)-5-({[tert-butyl(dimethyl)silyl]oxy}methyl)-1H-indol-2-yl]boronic acid (0.53 g, 1.3 eq., prepared as in Tetrahedron Lett. 2002, 43(15), 2695) and the protected iodotetrahydroindazole from the foregoing step (0.346 g, 1 eq.) were dissolved in DME (2.9 ml). 2N Na₂CO₃ (7.6 ml) was added and the reaction mixture was stirred for 0.5 h. Pd(PPh₃)₄ (freshly prepared, 65 mg) was then added and the mixture heated overnight at 85° C. On cooling, the reaction was diluted with brine and extracted with EtOAc (×3), dried (MgSO₄) and evaporated in vacuo. The residue was purified by flash column chromatography (silica gel; eluent 20-50% EtOAc/Hexane) to afford desired product (160 mg, note, selective mono-deprotection of Boc group).

### Scheme 3—Step 4

To a solution of the silyl ether from the foregoing step (130 mg) in MeCN (5 ml) was added triethylamine trihydrofluoride (0.2 ml, 3 eq.) and the reaction stirred at room temperature 4 hours. After this time, the mixture was diluted with EtOAc and washed with sodium bicarbonate solution (×2) and brine then dried (MgSO₄) and evaporated in vacuo. Used without further purification.

### Scheme 3—Step 5

To a solution of the alcohol from the foregoing step (100 mg) in DCM (4 ml) was added Dess-Martin periodinane (175 mg, 1.5 eq.) and the reaction stirred at room temperature 0.5 hours. After this time, the mixture was diluted with ether and washed with sodium thiosulfate solution, sodium bicarbonate solution and brine then dried (MgSO₄) and evaporated in vacuo. Yield 100 mg. Used without further purification.

### Scheme 3—Step 6

To a solution of the aldehyde from the foregoing step (45 mg) in 1,2-DCE (3 ml) was added morpholine (0.046 ml, 4 eq.) (shown using morpholine but any amine, e.g., piperidine, Boc(1,4-diaminocyclohexane etc. can be used in this step) and sodium triacetoxyborohydride (46 mg, 1.6 eq.) and the reaction stirred at room temperature 4 hours. After this time, the mixture was diluted with EtOAc and washed with sodium bicarbonate solution and brine then dried (MgSO₄) and evaporated in vacuo. The residue was purified by flash column chromatography (silica gel; eluent 5% MeOH/DCM) to afford desired product (40 mg).

### Scheme 3—Step 7

To a solution of the product from the foregoing step (40 mg) in DCM (2 ml) was added TFA (1 ml) and the reaction stirred at room temperature 2 hours. After this time, the mixture was evaporated in vacuo, and the residue purified by SCX cartridge then by flash column chromatography (silica gel; eluent 10% MeOH/DCM) to afford the desired product (16 mg).

### Scheme 4

### Scheme 4—Step 1:

To a solution of 4-(3-pyridyl)-cyclohexanone (5 g, 29 mmol., prepared as in J. Med. Chem. 1989, p 355) in THF (50 ml) was added ethyl formate (9 ml), NaH (1.3 g) and ethanol (5 drops). The mixture was refluxed for 3 h., cooled, diluted with ether and filtered. The solid was washed with further ether then dissolved in ethanol (200 ml) and treated with AcOH (2 eq.), hydrazine hydrate (1.2 eq.) and stirred at room temperature overnight. The solvent was evaporated and the residue taken up in EtOAc and washed with water and brine, then dried (MgSO₄) and evaporated in vacuo to afford the desired 5-(pyridin-3-yl)-4,5,6,7-tetrahydroindazole (2.75 g).

This intermediate was elaborated by the procedures of Scheme 3 to provide a variety of tetrahydroindazole derivatives.

Compounds obtained via Schemes 3 and 4 are listed in the following table:

| | | | Synthesis |
|---|---|---|---|
| Example | Structure | ms [MH+] | Scheme |
| 114 | 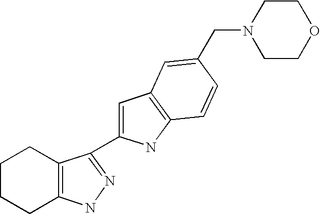 | 337 | 3 |
| 115 | 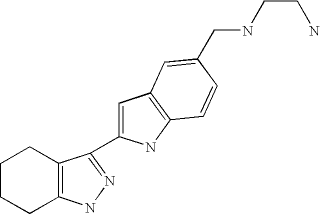 | 310 | 3 |
| 116 | 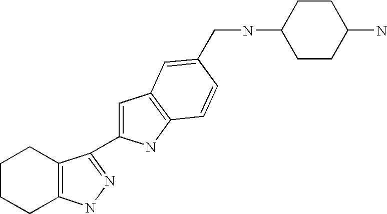 | 364 | 3 |
| 117 | 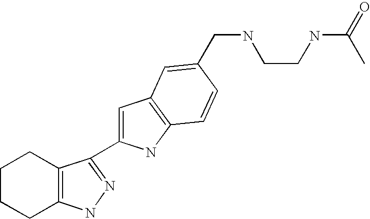 | 352 | 3 |
| 118 | 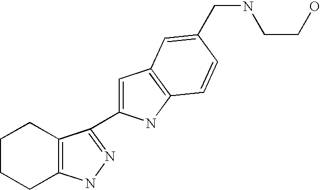 | 311 | 3 |
| 119 | 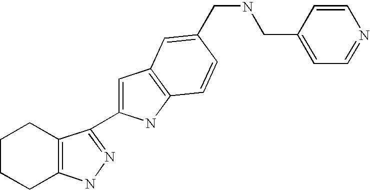 | 358 | 3 |
| 120 | 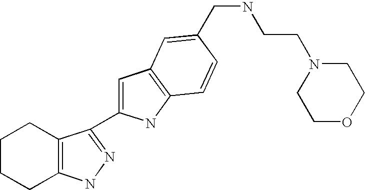 | 380 | 3 |
| 121 | 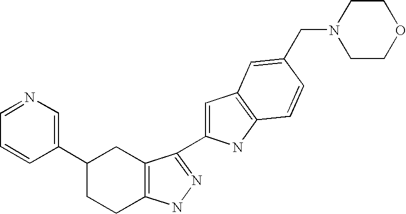 | 414 | 4 |
| 122 | 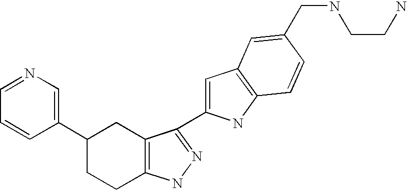 | 387 | 4 |
| 123 | 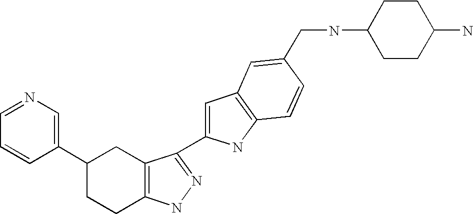 | 441 | 4 |
| 124 | 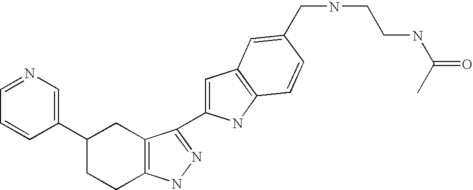 | 429 | 4 |
| 125 | 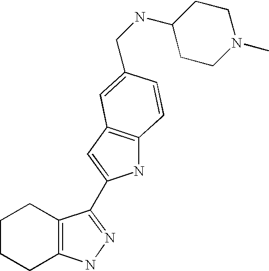 | 364 | 3 |
| 126 | 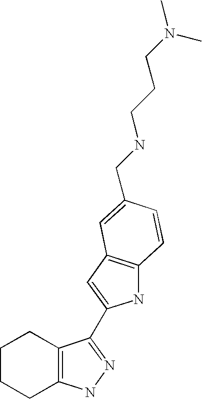 | 352 | 3 |
| 127 | 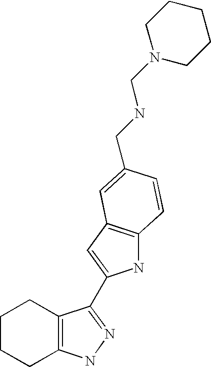 | 364 | 3 |
| 128 | 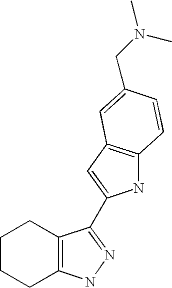 | 295 | 3 |
| 129 | 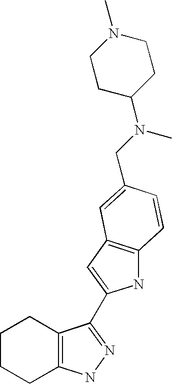 | 378 | 3 |
| 130 | 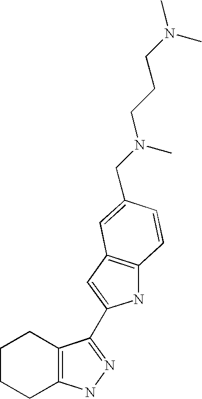 | 366 | 3 |

In the aforegoing tables, H atoms are to be inferred where unsatisfied valencies on heteroatoms are shown.

1. (canceled)

2. A method for treatment or prevention of a neurodegenerative disease associated with hyperphosphorylation of tau in a human patient, said method comprising administering to that patient an effective amount of a compound of formula IA or IB: 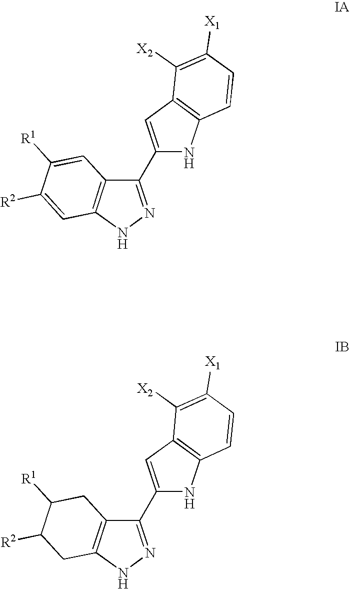
or a pharmaceutically acceptable salt or hydrate thereof, wherein:
one of R¹ and R² represents H, halogen or C₁₋₄alkyl and the other is selected from H, halogen CN, NO, CF₃, OR⁵, N(R⁵)₂, aryl which optionally bears up to 3 substituents selected from halogen CN, NO₂, CF₃, OR⁵, C₁₋₄alkyl, hydroxyC₁₋₄alkyl, CO₂R⁵ and CON(R⁵)₂, and non-aromatic hydrocarbon of up to 6 carbon atoms which is optionally substituted with halogen CN, CF₃, or OR⁵;
or in formula IB R¹ or R² may represent oxo;
one of X1 and X2 represents H and the other represents L-OR³ or L-NR³R⁴; where L represents a bond or an alkylene group of up to 4 carbon atoms which optionally bears an oxo substituent;
R³ represents H or nonaromatic hydrocarbon of up to 10 carbon atoms, optionally substituted with halogen, CN, CF₃, OR⁵, N(R⁵)₂ or NR⁵COC₁₋₄alkyl;
or R³ represents aryl, arylC₁₋₄alkyl, C-heterocyclyl or C-heterocyclylC₁₋₄alkyl, any of which optionally bears up to 3 substituents selected from halogen, CN, CF₃, OR⁵, C₁₋₄alkyl, aryl or arylC₁₋₄alkyl;
R⁴ represents H or C₁₋₄alkyl; or R³ and R⁴ together complete a mono- or bicyclic heterocyclic ring system of up to 10 members which optionally bears up to 3 substituents selected from halogen, CN, CF₃, C₁₋₄alkyl, oxo, OR⁵, N(R⁵)₂, (R⁵)₂, NC₁₋₄alkyl, COC₁₋₄alkyl, aryl, arylC₁₋₄alkyl, C-heterocyclyl and C-heterocyclylC₁₋₄alkyl, said aryl arylC₁₋₄alkyl C-heterocyclyl and C-heterocyclylC₁₋₄alkyl themselves optionally bearing up to 3 substituents selected from halogen, CN, CF₃, C₁₋₄alkyl and OR⁵;
R⁵ represents H or C₁₋₄alkyl, or two R⁵ groups attached to the same nitrogen atom may complete a heterocyclic ring of 5 or 6 members optionally bearing a substituent selected from halogen, oxo, CF₃ and C₁₋₄alkyl;
where “aryl” refers to phenyl, naphthyl or optionally benzofused 5- or 6-membered heteroaryl, and “C-heterocyclyl” refers to a 5- or 6-membered nonaromatic ring in which the attachment point is a carbon atom and in which from 1 to 3 of the ring atoms are independently selected from N, O and S.

3. The method according to claim 2 wherein said neurodegenerative disease associated with hyperphosphorylation of tau is selected from Alzheimer's disease (AD), frontotemporal dementia, Pick's disease and parkinsonism linked to chromosome 17 (FTDP-17).

4. The method according to claim 2 wherein one of X1 and X2 represents H and the other represents CH₂NR³R⁴.

5. The method according to claim 2 wherein the compound is a compound of formula II: 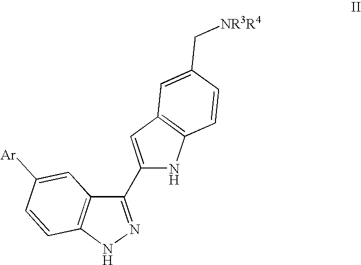
or a pharmaceutically acceptable salt or hydrate thereof;
wherein:
Ar represents phenyl, naphthyl or optionally benzofused 5- or 6-membered heteroaryl, any of which optionally bears up to 3 substituents selected from halogen, CN, NO₂, CF₃, OR⁵, C₁₋₄alkyl, hydroxyC₁₋₄alkyl, CO₂R⁵ and CON(R⁵)₂;
R³ represents H or nonaromatic hydrocarbon of up to 10 carbon atoms, optionally substituted with halogen, CN, CF₃, OR⁵, N(R⁵)₂, or NR⁵COC₁₋₄alkyl;
or R³ represents aryl, arylC₁₋₄alkyl, C-heterocyclyl or C-heterocyclylC₁₋₄alkyl, any of which optionally bears up to 3 substituents selected from halogen, CN, CF₃, C₁₋₄alkyl, aryl or arylC₁₋₄alkyl;
R⁴ represents H or C₁₋₄alkyl; or R³ and R⁴ together complete a mono- or bicyclic heterocyclic ring system of up to 10 members which optionally bears up to 3 substituents selected from halogen, CN, CF₃, C₁₋₄alkyl, oxo, OR⁵, N(R⁵)₂, (R⁵)₂NC₁₋₄alkyl, COC₁₋₄alkyl, aryl, arylC₁₋₄alkyl, C-heterocyclyl and C-heterocyclylC₁₋₄alkyl, said aryl, arylC₁₋₄alkyl, C-heterocyclyl and C-heterocyclylC₁₋₄alkyl themselves optionally bearing up to 3 substituents selected from halogen, CN, CF₃, C₁₋₄alkyl and OR⁵;
R⁵ represents H or C₁₋₄alkyl, or two R⁵ groups attached to the same nitrogen atom may complete a heterocyclic ring of 5 or 6 members optionally bearing a substituent selected from halogen, oxo, CF₃ and C₁₋₄alkyl;
where “aryl” refers to phenyl, naphthyl or optionally benzofused 5- or 6-membered heteroaryl, and “C-heterocyclyl” refers to a 5- or 6-membered nonaromatic ring in which the attachment point is a carbon atom and in which from 1 to 3 of the ring atoms are independently selected from N, O and S.

6. A compound of formula IB: 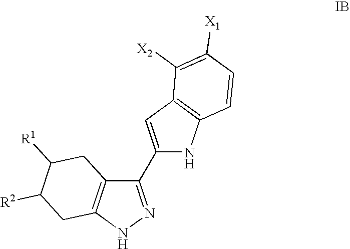
or a pharmaceutically acceptable salt or hydrate thereof: wherein:
one of R¹ and R² represents H, halogen or C₁alkyl and the other is selected from H, halogen, CN, NO₂, CF₃, OR⁵, N(R⁵)₂, aryl which optionally bears up to 3 substituents selected from halogen, CN, NO₂, CF₃, OR⁵, C₁₋₄alkyl, hydroxyC₁₋₄alkyl, CO₂R⁵ and CON(R⁵)₂, and non-aromatic hydrocarbon of up to 6 carbon atoms which is optionally substituted with halogen, CN, CF₃ or OR⁵;
or in formula IB R¹ or R² may represent oxo;
one of X1 and X2 represents H and the other represents L-OR³ or L-NR³R⁴; where L represents a bond or an alkylene group of up to 4 carbon atoms which optionally bears an oxo substituent;
R³ represents H or nonaromatic hydrocarbon of up to 10 carbon atoms, optionally substituted with halogen, CN, CF₃, OR⁵, N(R⁵)₂, or NR⁵COC₁₋₄alkyl;
or R³ represents aryl, arylC₁₋₄alkyl, C-heterocyclyl or C-heterocyclylC₁₋₄alkyl, any of which optionally bears up to 3 substituents selected from halogen, CN, CF₃, OR⁵, C₁₋₄alkyl, aryl or arylC₁₋₄alkyl;
R⁴ represents H or C₁₋₄alkyl; or R³ and R⁴ together complete a mono- or bicyclic heterocyclic ring system of up to 10 members which optionally bears up to 3 substituents selected from halogen, CN, CF₃, C₁₋₄alkyl, oxo, OR⁵, N(R⁵)₂, (R⁵)₂NC₁₋₄alkyl, COC₁₋₄alkyl, aryl, arylC₁₋₄alkyl, C-heterocyclyl and C-heterocyclylC₁₋₄alkyl, said aryl, arylC₁₋₄alkyl, C-heterocyclyl and C-heterocyclylC₁₋₄alkyl themselves optionally bearing up to 3 substituents selected from halogen, CN, CF₃, C₁₋₄alkyl and OR⁵;
R⁵ represents H or C₁₋₄alkyl, or two R⁵ groups attached to the same nitrogen atom may complete a heterocyclic ring of 5 or 6 members optionally bearing a substituent selected from halogen, oxo, CF₃ and C₁₋₄alkyl;
where “aryl” refers to phenyl, naphthyl or optionally benzofused 5- or 6-membered heteroaryl, and “C-heterocyclyl” refers to a 5- or 6-membered nonaromatic ring in which the attachment point is a carbon atom and in which from 1 to 3 of the ring atoms are independently selected from N, O and S.

7. A compound of formula II: 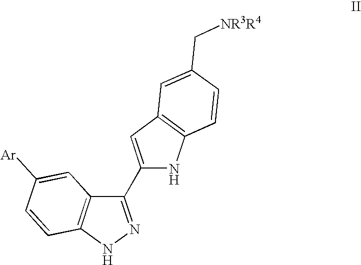
or a pharmaceutically acceptable salt or hydrate thereof;
wherein:
Ar represents phenyl, naphthyl or optionally benzofused 5- or 6-membered heteroaryl, any of which optionally bears up to 3 substituents selected from halogen, CN, NO₂, CF₃, OR⁵, C₁₋₄alkyl, hydroxyC₁₋₄alkyl, CO₂R⁵ and CON(R⁵)₂;
R³ represents H or nonaromatic hydrocarbon of up to 10 carbon atoms, optionally substituted with halogen, CN, CF₃, OR⁵, N(R⁵)₂ or NR⁵COC₁₋₄alkyl;
or R³ represents aryl arylC₁₋₄alkyl, C-heterocyclyl or C-heterocyclylC₁₋₄alkyl, any of which optionally bears up to 3 substituents selected from halogen, CN, CF₃, OR⁵, C₁₋₄alkyl, aryl or arylC₁₋₄alkyl;
R⁴ represents H or C₁₋₄alkyl; or R³ and R⁴ together complete a mono- or bicyclic heterocyclic ring system of up to 10 members which optionally bears up to 3 substituents selected from halogen, CN, CF₃, C₁₋₄alkyl, oxo, OR⁵, N(R⁵)₂, (R⁵)₂NC₁₋₄alkyl, COC₁₋₄alkyl, aryl arylC₁₋₄alkyl, C-heterocyclyl and C-heterocyclylC₁₋₄alkyl, said aryl arylC₁₋₄alkyl, C-heterocyclyl and C-heterocyclylC₁₋₄alkyl themselves optionally bearing up to 3 substituents selected from halogen, CN, CF₃, C₁₋₄alkyl and OR⁵;
R⁵ represents H or C₁₋₄alkyl, or two R⁵ groups attached to the same nitrogen atom may complete a heterocyclic ring of 5 or 6 members optionally bearing a substituent selected from halogen, oxo, CF₃ and C₁₋₄alkyl;
where “aryl” refers to phenyl naphthyl or optionally benzofused 5- or 6-membered heteroaryl, and “C-heterocyclyl” refers to a 5- or 6-membered nonaromatic ring in which the attachment point is a carbon atom and in which from 1 to 3 of the ring atoms are independently selected from N, O and S.

8. A compound according to claim 7 wherein NR³R⁴ takes the form: 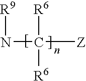
where n is 1, 2, 3 or 4;
Z represents OR⁷ or NR⁷R⁸;
each R⁶ independently represents H, or together with R⁷ represents the atoms necessary to complete a 5- or 6-membered heterocyclic ring, or together with R⁹ represents the atoms necessary to complete a 5- or 6-membered heterocyclic ring, or two R⁶ groups may together represent the atoms necessary to complete a 5- or 6-membered carbocyclic ring;
R⁷ represents H or C₁₋₄alkyl, or together with an R⁶ group represents the atoms necessary to complete a 5- or 6-membered heterocyclic ring, or together with R⁹ represents the atoms necessary to complete a 5- or 6-membered heterocyclic ring;
R⁸ represents H, C₁₋₄alkyl or COC₁₋₄alkyl; or R⁷ and R⁸ may complete a heterocyclic ring of 5 or 6 members optionally bearing a substituent selected from halogen, oxo, CF₃ and C₁₋₄alkyl; and
R⁹ represents H or C₁₋₄alkyl, or together with an R⁶ group represents the atoms necessary to complete a 5- or 6-membered heterocyclic ring, or together with R⁷ represents the atoms necessary to complete a 5- or 6-membered heterocyclic ring;
provided that when R⁷ and R⁹ complete a ring, n is 2 and all four R⁶ groups are H;
and that when n is 1, both the R⁶ groups are H.

9. A compound according to claim 8 wherein Z is NR⁷R⁸.

10. A pharmaceutical composition comprising a compound according to claim 6 or a pharmaceutically acceptable salt or hydrate thereof and a pharmaceutically acceptable carrier.

11. (canceled)

12. A pharmaceutical composition comprising a compound according to claim 7 or a pharmaceutically acceptable salt or hydrate thereof and a pharmaceutically acceptable carrier.
